(19) United States
(12) Patent Application Publication
(10) Pub. No.: US 2018/0277252 A1
(43) Pub. Date: Sep. 27, 2018

(54) Person Engagement Index for Providing Automated Personalized Healthcare Functions

(71) Applicant: GetWellNetwork, Inc., Bethesda, MD (US)

(72) Inventors: Karen Drenkard, Fairfax, VA (US); Ellen Swartwout, Galloway, NJ (US); Michael J. O'Neil, Bethesda, MD (US); Stephen Rowe, Brooklyn, NY (US)

(21) Appl. No.: 15/923,402

(22) Filed: Mar. 16, 2018

Related U.S. Application Data
(60) Provisional application No. 62/472,902, filed on Mar. 17, 2017.

(51) Int. Cl. G16H 50/30; G16H 10/60; G16H 40/63

(52) U.S. Cl. CPC G16H 50/30; G06F 3/0482; G16H 40/63; G16H 10/60

(57) ABSTRACT

Aspects of the disclosure relate to implementing and using a data processing system with a person engagement index to provide automated personalized healthcare functions. In some embodiments, a computing platform may receive information about a patient or other healthcare-consuming person from several sources including electronic medical records, healthcare providers, the person, and other sources. Based on the information, a person engagement index measuring the person's capacity to engage in healthcare may be generated by the computing platform. In addition to being calculated by the computing platform based on medical records and other such information, the person engagement index may be calculated by the computing platform based on answers to survey questions, as well as patient interaction information indicating how the person interacts with personalized educational content. The person engagement index may be used by the computing platform to provide personalized care according to an interactive care model in various contexts.

### CROSS-REFERENCE TO RELATED APPLICATIONS

This application claims the benefit of and priority to U.S. Provisional Patent Application Ser. No. 62/472,902, filed Mar. 17, 2017, and entitled “Person Engagement Index For Providing Automated Personalized Healthcare Functions,” which is incorporated by reference herein in its entirety.

### BACKGROUND

Aspects of the disclosure relate to electrical computers, data processing systems, and computing platforms for providing automated personalized healthcare functions. In particular, one or more aspects of the disclosure relate to implementing and using a data processing system with a person engagement index to provide automated personalized healthcare functions.

### SUMMARY

Aspects of the disclosure provide effective, efficient, scalable, and convenient technical solutions that address and overcome the technical problems associated with providing personalized healthcare solutions. In particular, one or more aspects of the disclosure provide techniques for implementing and using a data processing system with a person engagement index to provide automated personalized healthcare functions.

Some aspects of the disclosure relate to providing automated personalized healthcare functions. In some instances, a computing platform may receive information about a patient or other healthcare-consuming person from several sources including electronic medical records, healthcare providers, the person herself or himself, and other sources. Based on the information, a person engagement index measuring the person's capacity to engage in healthcare may be generated by the computing platform. In addition to being calculated by the computing platform based on medical records and other such information, the person engagement index may be calculated by the computing platform based on answers to survey questions provided by the person, as well as patient interaction information indicating how the person interacts with personalized educational content provided to the person based on the person's symptoms, conditions, and other such aspects of patient care.

In some instances, the person engagement index may be used (e.g., by the computing platform) to provide personalized care according to an interactive care model in inpatient and/or outpatient contexts. The computing platform may use the person engagement index to provide automated treatment recommendations and effectiveness ratings. Such recommendations may include recommendations to adjust one or more of a patient rounding schedule, a patient medication schedule, and a patient reminder schedule. Furthermore, effectiveness ratings of original and/or modified patient rounding schedules, patient medication schedules, and patient reminder schedules may be determined by the computing platform and displayed so that clinicians may learn how to best treat the person (who may, e.g., be a patient in a medical setting) as an individual.

In some embodiments, a computing platform having at least one processor, a memory, and a communication interface may receive first data associated with an engagement in healthcare factor for a patient. Next, the computing platform may receive second data associated with a technology use in healthcare factor for the patient. Subsequently, the computing platform may receive third data associated with a proactive approach to healthcare factor for the patient. Next, the computing platform may receive fourth data associated with a psychosocial support for healthcare factor for the patient. Subsequently, the computing platform may determine a person engagement index (PEI) score for the patient based on the first data, the second data, the third data, and the fourth data. Finally, the computing platform may output the PEI score for the patient to a display device.

In some embodiments, the first data may comprise fifth data associated with a safety involvement sub-factor for the patient, sixth data associated with a health literacy sub-factor for the patient, seventh data associated with a preventative actions sub-factor for the patient, and eighth data associated with a patient preferences sub-factor for the patient. In some embodiments, the third data may comprise ninth data associated with an activation/motivation sub-factor for the patient, tenth data associated with a patient preferences sub-factor for the patient, and eleventh data associated with a psychosocial support sub-factor for the patient.

In some embodiments, the first data, the second data, the third data, and/or the fourth data may comprise answers to a plurality of survey questions. In some embodiments, the first data, the second data, the third data, and/or the fourth data may comprise patient demographic information. In some embodiments, the first data, the second data, the third data, and/or the fourth data may comprise interaction information indicating one or more patient interactions with educational content.

In some embodiments, the computing platform may select, from a database, based on the PEI score, educational content for provisioning to the patient, and transmit the selected educational content to a mobile device associated with the patient. In some embodiments, the computing platform may determine a patient treatment plan based on the PEI score associated with the patient, and output the patient treatment plan to the display device. In some embodiments, the patient treatment plan comprises a patient medication plan.

In some embodiments, the computing platform may determine a preferred contact method for contacting the patient based on the PEI score associated with the patient, and output the preferred contact method to the display device. In some embodiments, the computing platform may determine, based on the PEI score associated with the patient, an effectiveness of a patient treatment plan, and output the effectiveness of the patient treatment plan to the display device.

In some embodiments, a computing platform having at least one processor, a memory, and a communication interface may receive, via a portable survey tool, a plurality of survey responses from a patient. The plurality of survey responses may comprise first responses indicative of an engagement in healthcare factor for the patient, second responses indicative of a technology use in healthcare factor for the patient, third responses indicative of a proactive approach to healthcare factor for the patient, and fourth responses indicative of a psychosocial support for healthcare factor for the patient. Next, the computing platform may determine a person engagement index (PEI) score for the patient based on the plurality of survey responses received via the portable survey tool. Finally, the computing platform may output the PEI score for the patient to one or more output devices.

In some embodiments, the portable survey tool may comprise a mobile application running on a mobile device associated with the patient. In some embodiments, the portable survey tool may comprise a web-based interface provided by a website. In some embodiments, the computing platform may transmit, to a mobile device associated with the patient, a link for accessing the portable survey tool.

In some embodiments, the computing platform may receive, via the portable survey tool, at least the first responses, determine that one or more of the second responses, the third responses, and the fourth responses have not been received, and transmit, to a mobile device associated with the patient, a link to a website configured to prompt the patient to provide the one or more responses that have not been received.

In some embodiments, a computing platform having at least one processor, a memory, and a communication interface may receive, from an inpatient provider management console, a patient admission notification indicating that a patient has been admitted to the care of a healthcare provider. Next, responsive to receiving the patient admission notification, the computing platform may identify a person engagement index (PEI) score associated with the patient. Subsequently, the computing platform may generate one or more notifications for the inpatient provider management console based on the PEI score associated with the patient. Finally, the computing platform may send the one or more notifications to the inpatient provider management console, and the one or more notifications may instruct the healthcare provider to perform specific actions based on the PEI score associated with the patient.

In some embodiments, the computing platform may determine, based on the PEI score associated with the patient, a reminder frequency for reminding the patient to follow a patient treatment plan. The one or more notifications may comprise a notification indicating that the patient should be reminded to follow the patient treatment plan. In some embodiments, the computing platform may determine, based on the PEI score associated with the patient, an effectiveness of a patient treatment plan. The one or more notifications may comprise a notification indicating the effectiveness of the patient treatment plan. In some embodiments, the computing platform may transmit, via the communication interface, a patient identifier to an electronic medical records (EMR) system, and may receive, from the EMR system, the PEI score associated with the patient.

In some embodiments, a computing platform having at least one processor, a memory, and a communication interface may receive, from a patient bedside device, a session initiation request linking the patient bedside device to a patient record associated with a patient. Next, responsive to receiving the session initiation request, the computing platform may identify a person engagement index (PEI) score associated with the patient. Subsequently, the computing platform may generate one or more configuration commands for the patient bedside device based on the PEI score associated with the patient. Finally, the computing platform may send the one or more configuration commands to the patient bedside device. The one or more configuration commands may direct the patient bedside device to selectively enable or disable one or more functions of the patient bedside device based on the PEI score associated with the patient.

In some embodiments, the computing platform may receive patient information associated with the patient, and determine the PEI score based on the patient information. The patient information may comprise first data associated with an engagement in healthcare factor for the patient, second data associated with a technology use in healthcare factor for the patient, third data associated with a proactive approach to healthcare factor for the patient, and fourth data associated with a psychosocial support for healthcare factor for the patient. The patient information may additionally or alternatively comprise patient demographic information. The computing platform may determine the PEI score based on a comparison of the patient demographic information with demographic information for other patients different from the patient.

In some embodiments, the one or more configuration commands may direct the patient bedside device to selectively enable or disable bedside device treatment notifications. In some embodiments, the computing platform may generate, based on the PEI score associated with the patient, a medication plan for the patient. In addition, the bedside device treatment notifications may comprise reminders to take one or more medications according to the medication plan. In some embodiments, the computing platform may receive, from a survey tool, answers to the one or more questions, and may update, based on the answers, the PEI score.

In some embodiments, one or more configuration commands may direct the patient bedside device to selectively enable or disable access to one or more patient treatment videos. In some embodiments, the computing platform may receive, from the patient bedside device, patient viewing history information associated with the one or more patient treatment videos, and update the PEI score based on the patient viewing history information. The patient viewing history information may comprise at least one of an order of selection, by the patient, of the one or more patient treatment videos, start times at which the patient started watching the one or more patient treatment videos, or stop times at which the patient stopped watching the one or more patient treatment videos.

In some embodiments, responsive to the PEI score exceeding a threshold, the computing platform may select detailed patient treatment information associated with the patient. The one or more configuration commands may direct the patient bedside device to selectively enable or disable access to the detailed patient treatment information.

These features, along with many others, are discussed in greater detail below.

### BRIEF DESCRIPTION OF THE DRAWINGS

The present disclosure is illustrated by way of example and not limited in the accompanying figures in which like reference numerals indicate similar elements and in which:

FIG. 1 depicts an illustrative computing environment for implementing and using a data processing system with a person engagement index to provide automated personalized healthcare functions in accordance with one or more example embodiments;

FIGS. 2A-2F depict an illustrative event sequence for implementing and using a data processing system with a person engagement index to provide automated personalized healthcare functions in accordance with one or more example embodiments;

FIGS. 3A-3E depict an illustrative event sequence for implementing and using a data processing system with a person engagement index to provide automated personalized healthcare functions in accordance with one or more example embodiments;

FIGS. 4, 5, 6, 7A-7D, 8A-8E, and 9A-9E illustrate example user interfaces and user experience workflows for implementing and using a data processing system with a person engagement index to provide automated personalized healthcare functions in accordance with one or more example embodiments;

FIGS. 10-13 illustrate example methods for implementing and using a data processing system with a person engagement index to provide automated personalized healthcare functions in accordance with one or more example embodiments.

### DETAILED DESCRIPTION

In the following description of various illustrative embodiments, reference is made to the accompanying drawings, which form a part hereof, and in which is shown, by way of illustration, various embodiments in which aspects of the disclosure may be practiced. It is to be understood that other embodiments may be utilized, and structural and functional modifications may be made, without departing from the scope of the present disclosure.

It is noted that various connections between elements are discussed in the following description. It is noted that these connections are general and, unless specified otherwise, may be direct or indirect, wired or wireless, and that the specification is not intended to be limiting in this respect.

Some aspects of the disclosure relate to providing automated personalized healthcare functions. In some instances, a computing platform may receive information about a patient or other healthcare-consuming person from several sources including electronic medical records, clinicians, other healthcare providers and/or staff, the person himself or herself, and other sources. Based on the information, a person engagement index measuring the person's capacity to engage in healthcare may be generated by the computing platform. In addition to being calculated by the computing platform based on medical records and other such information, the person engagement index may be calculated by the computing platform based on answers to survey questions provided by the person, as well as patient interaction information indicating how the person interacts with personalized educational content provided to the person based on the person's symptoms, conditions, and other such aspects of patient care.

In some instances, the person engagement index may be used (e.g., by the computing platform) to provide personalized care according to an interactive care model in inpatient and/or outpatient contexts. The computing platform may use the person engagement index to provide automated treatment recommendations and effectiveness ratings. Such recommendations may include recommendations to adjust one or more of a patient rounding schedule, a patient medication schedule, and a patient reminder schedule. Furthermore, effectiveness ratings of original and/or modified patient rounding schedules, patient medication schedules, and patient reminder schedules may be determined by the computing platform and displayed so that clinicians may learn how to best treat the person (who may, e.g., be a patient in a medical setting) as an individual.

Various aspects of determining a person engagement index and using the person engagement index to provide automated personalized healthcare functions are described in detail below.

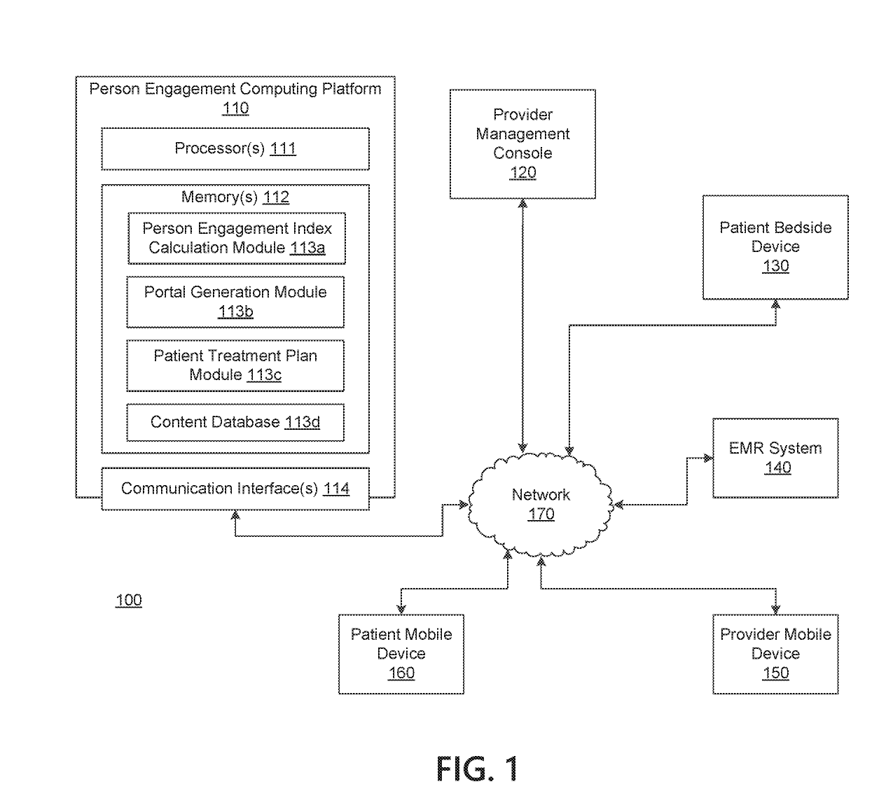

FIG. 1 depicts an illustrative computing environment for implementing and using a data processing system with a person engagement index to provide automated personalized healthcare functions in accordance with one or more example embodiments. Referring to FIG. 1, computing environment 100 may include one or more computing devices and/or other computer systems. For example, computing environment 100 may include a person engagement computing platform 110, a provider management console 120, a patient bedside device 130, an electronic medical records (EMR) system 140, a provider mobile device 150, and a patient mobile device 160.

The person engagement computing platform 110 may be configured to calculate and/or determine a person engagement index to provide automated personalized healthcare functions, as discussed in greater detail below. In some instances, person engagement computing platform 110 may determine a person engagement index (PEI) score for a patient or other person, generate and provide a personalized patient portal for delivery to a device associated with the patient, facilitate one or more modifications to a personalized patient treatment plan, and select educational interactive content for provision to a patient. In some embodiments, to generate and provide the patient portal, person engagement computing platform 110 may be and/or include a web server configured to host a web-based patient portal. In some embodiments, person engagement computing platform 110 may be configured to generate and render various user interfaces for accessing the personalized healthcare functions. It should be understood that, throughout this disclosure, where person engagement computing platform 110 is described as determining a PEI score and/or providing other functions with respect to a patient, person engagement computing platform 110 may similarly determine a PEI score and/or provide such other functions with respect to a person who is not a patient, such as a non-patient healthcare consumer or other person interacting with person engagement computing platform 110 and/or other systems in computing environment 100 in an ambulatory clinic setting, in a home-use setting, and/or in other settings (e.g., where, for instance, the person might not be considered a “patient” in the traditional sense of the word).

The provider management console 120 may be configured to provide management functions for one or more clinicians, clinical staff, or healthcare providers, and provider management console 120 may be configured to interact with the person engagement computing platform 110 to determine and execute a patient treatment plan. The patient bedside device 130 may be configured to store, update, and/or maintain one or more applications for interacting with a patient portal, receive patient inputs, display education content, and track patient interactions with the educational content. In some embodiments, the patient bedside device 130 may be a device owned by the patient. In such embodiments, patient bedside device 130 may perform the functions associated with both the patient bedside device 130 and the patient mobile device 160. EMR system 140 may be configured to maintain and/or retrieve one or more electronic medical records associated with a patient. The electronic medical records may include information about the patient, including information relating to one or more previous visits to a healthcare provider associated with the EMR system 140 and/or other healthcare providers. Provider mobile device 150 may be any mobile device owned by or associated with one or more clinicians and/or clinical staff of a healthcare provider. Similarly, patient mobile device 160 may be any mobile device owned by or associated with a patient. The provider mobile device 150 and/or patient mobile device 160 may be provided by a healthcare provider, or may be personally-owned devices.

In one or more arrangements, person engagement computing platform 110, provider management console 120, patient bedside device 130, EMR system 140, provider mobile device 150, and patient mobile device 160 may be any type of computing device capable of hosting and/or executing processes and services, transmitting and receiving information via a network, and providing interfaces to provide information to other such devices and receive information from other such devices. For example, person engagement computing platform 110, provider management console 120, patient bedside device 130, EMR system 140, provider mobile device 150, and patient mobile device 160 may, in some instances, be and/or include server computers, desktop computers, laptop computers, tablet computers, smart phones, or the like that may include one or more processors, memories, communication interfaces, storage devices, and/or other components. As illustrated in greater detail below, any and/or all of person engagement computing platform 110, provider management console 120, patient bedside device 130, EMR system 140, provider mobile device 150, and patient mobile device 160 may, in some instances, be special-purpose computing devices configured to perform specific functions.

Computing environment 100 also may include one or more computing platforms. For example, and as noted above, computing environment 100 may include person engagement computing platform 110. As illustrated in greater detail below, person engagement computing platform 110 may include one or more computing devices configured to perform one or more of the functions described herein. For example, person engagement computing platform 110 may include one or more computers (e.g., laptop computers, desktop computers, servers, server blades, or the like).

Computing environment 100 also may include one or more networks, which may interconnect one or more of person engagement computing platform 110, provider management console 120, patient bedside device 130, EMR system 140, provider mobile device 150, and patient mobile device 160. For example, computing environment 100 may include network 170. Network 170 may include one or more sub-networks (e.g., local area networks (LANs), wide area networks (WANs), or the like). Network 170 may be associated with a particular healthcare provider (e.g., a hospital, clinic, medical office or the like) and may interconnect one or more computing devices associated with the organization. For example, person engagement computing platform 110, provider management console 120, patient bedside device 130, EMR system 140, provider mobile device 150, and patient mobile device 160 may be associated with a hospital, and network 170 may be associated with and/or operated by the hospital, and may include one or more networks (e.g., LANs, WANs, virtual private networks (VPNs), or the like) that interconnect person engagement computing platform 110, provider management console 120, patient bedside device 130, EMR system 140, provider mobile device 150, and patient mobile device 160 and one or more other computing devices and/or computer systems that are used by, operated by, and/or otherwise associated with the organization. Network 170 may further connect to one or more public networks. For example, provider mobile device 150 and/or patient mobile device 160 might not be associated with a hospital, and may connect to network 170 via one or more public networks (e.g., the internet).

The person engagement computing platform 110 may include one or more processors 111, memory 112, and communication interface 114. A data bus may interconnect processor(s) 111, memory 112, and communication interface 114. Communication interface 114 may be a network interface configured to support communication between person engagement computing platform 110 and network 170. Memory 112 may include one or more program modules having instructions that when executed by processor(s) 111 cause the person engagement computing platform 110 to perform one or more functions described herein and/or one or more databases that may store and/or otherwise maintain information which may be used by such program modules and/or processor(s) 111. In some instances, the one or more program modules and/or databases may be stored by and/or maintained in different memory units of the person engagement computing platform 110 and/or by different computing devices that may form and/or otherwise make up the person engagement computing platform 110. For example, memory 112 may have, store, and/or include a person engagement index calculation module 113a, a portal generation module 113b, a patient treatment plan module 113c, and a content database 113d. The person engagement index calculation module 113a may calculate a person engagement index (PEI) score based on patient information including a patient's answers to survey questions, patient demographic information, and interaction information indicating a patient's interactions with one or more educational content items. The portal generation module 113b may generate a patient portal for display to a patient (e.g., via a patient bedside device 130 and/or a patient mobile device 160), and the patient portal may allow the patient to interact with the person engagement computing platform 110 (e.g., to take a survey for calculating the PEI score, to interact with educational content, and the like). The patient treatment plan module 113c may provide one or more recommendations and/or effectiveness ratings for personalizing a patient treatment plan based on the PEI score. Content database 113d may store interactive educational content for provision to a patient (e.g., via the patient portal).

A person engagement index may measure an engagement of a person with health care and/or health care services. The PEI score may include several factors measuring different aspects of engagement, including an engagement in healthcare factor, a technology use in healthcare factor, a proactive approach to healthcare factor, and a psychosocial support for healthcare factor. An engagement in healthcare factor may measure a person's beliefs and/or motivations about the importance of healthcare, understanding healthcare, and/or goals with respect to healthcare. For example, an engagement in healthcare factor may predict a person's willingness to research and seek out information regarding different conditions, treatments, and procedures related to healthcare. A technology use in healthcare factor may measure a person's ability and/or willingness to use technology to engage in healthcare. For example, an engagement in healthcare factor may predict a person's ability and/or willingness to use modern technology (e.g., smartphones, the Internet, etc.) to receive information about their healthcare, interact with medical providers, and otherwise engage with healthcare. A proactive approach to healthcare factor may measure a person's willingness to seek help for a health problem or health issue. For example, a proactive approach to healthcare factor may predict whether a person will contact a healthcare provider, a relative, or a friend when they are experiencing pain or other health issues. A proactive approach to healthcare factor additionally or alternatively may be associated with preventative care and/or maintaining health as related to a particular person. A psychosocial support for healthcare factor may measure a person's reliance on family and friends for support while dealing with health issues. For example, a psychosocial support for healthcare factor may predict whether a person can be reached and/or monitored through relatives or friends and/or whether a person believes that others will support him or her.

The engagement in healthcare factor may further include several sub-factors pertaining to various engagement domains, including a safety involvement sub-factor, a health literacy sub-factor, a preventative actions sub-factor, and a patient preferences sub-factor. A safety involvement sub-factor may measure a person's focus on safety and/or risk with respect to healthcare. A health literacy sub-factor may measure a person's knowledge in relation to healthcare and/or a person's willingness to acquire knowledge in relation to healthcare. A preventative actions sub-factor may measure a person's behaviors and/or habits with regard to health conditions and preventing health problems. A patient preferences sub-factor may measure a person's preferences for acquiring information about healthcare, participating in decisions about healthcare, and other aspects of engagement in healthcare.

The proactive approach to healthcare factor may further include several sub-factors pertaining to various engagement domains, including an activation/motivation sub-factor, a patient preferences sub-factor, and a psychosocial support sub-factor. An activation/motivation sub-factor may measure a person's involvement in healthcare, confidence in managing healthcare, and readiness to change with regard to health and treatments. A patient preferences sub-factor may measure a person's preferences regarding self-managing care and seeking assistance for health issues. A psychosocial support factor may measure a person's ability to proactively seek help from their support network.

The PEI score may be expressed as a value falling within a particular scale (e.g., 0 to 100). Using a patient treatment plan, healthcare may be tailored and personalized for the individual person based on his or her PEI score.

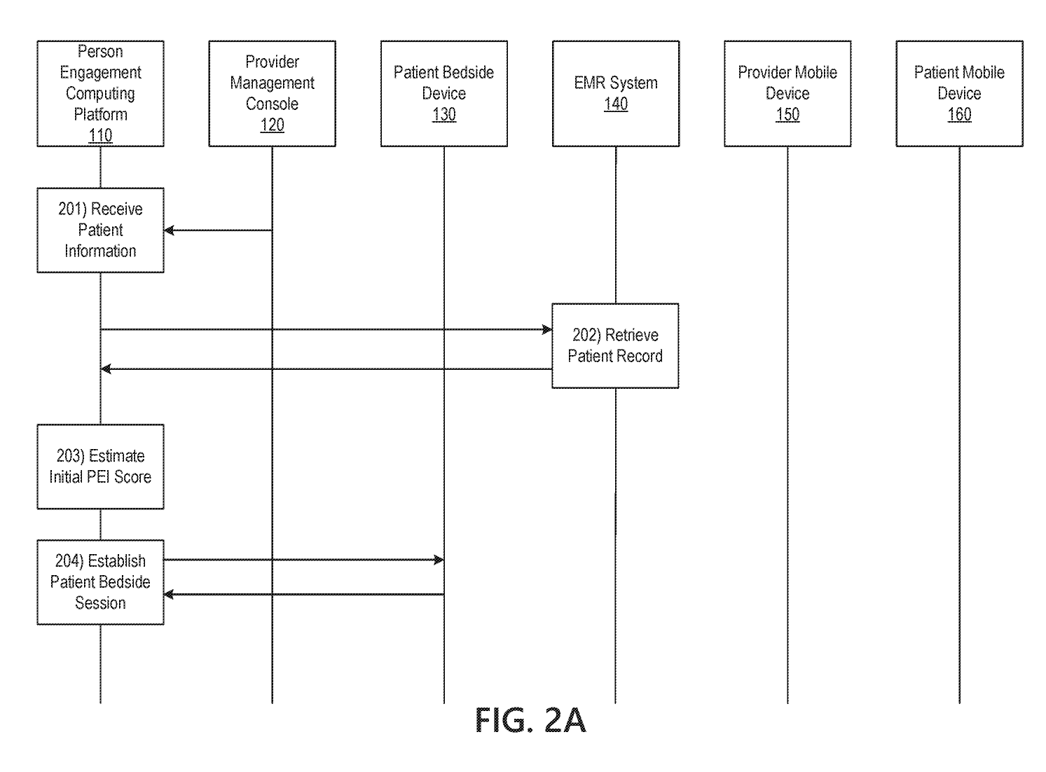

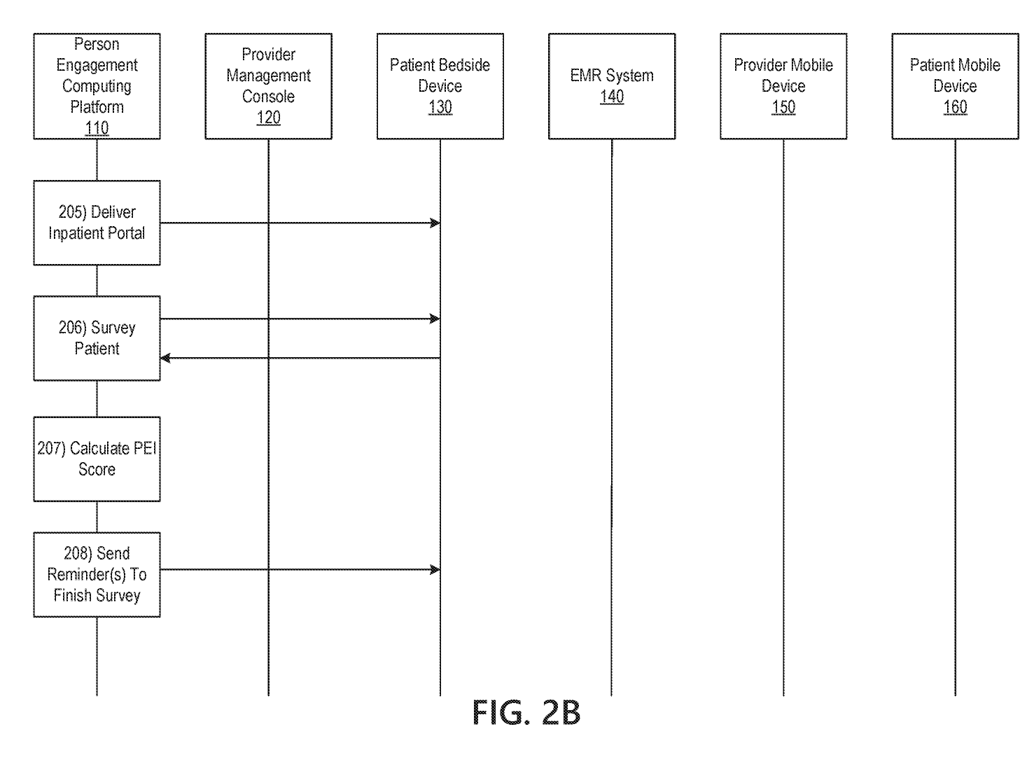

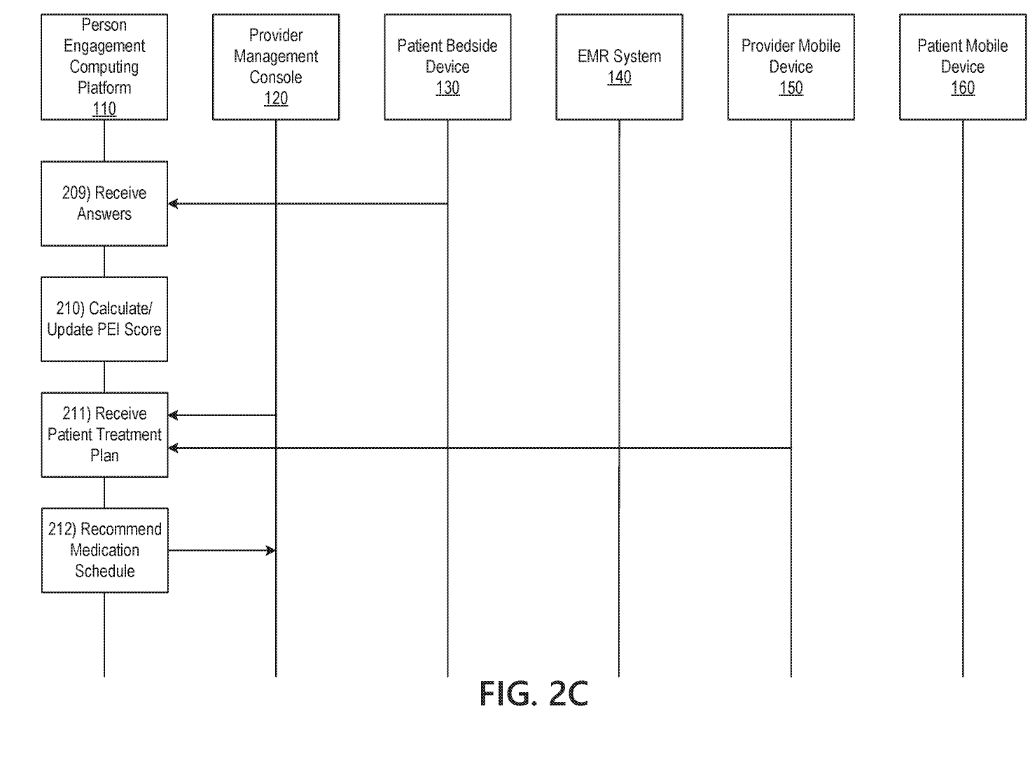

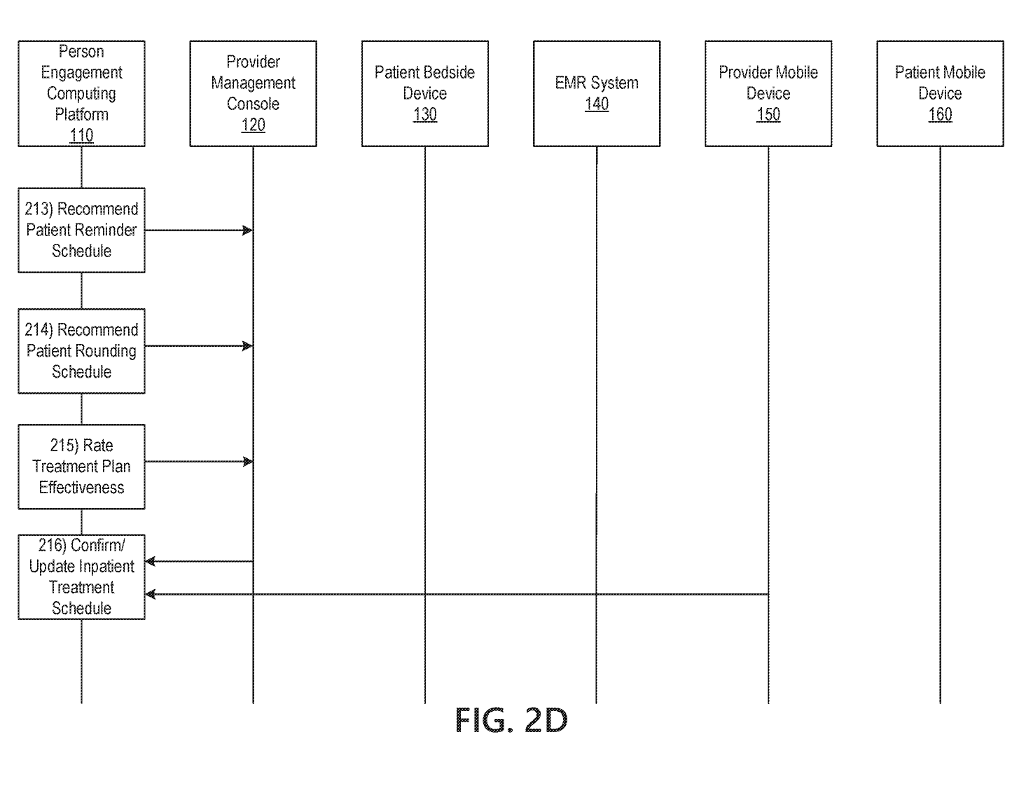

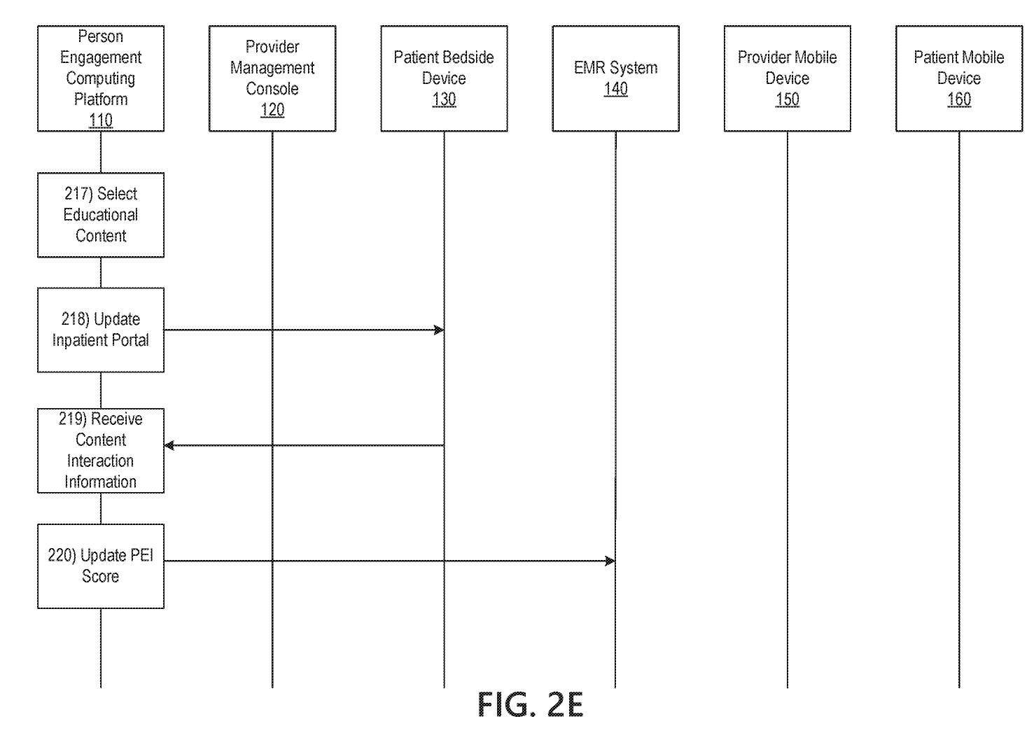

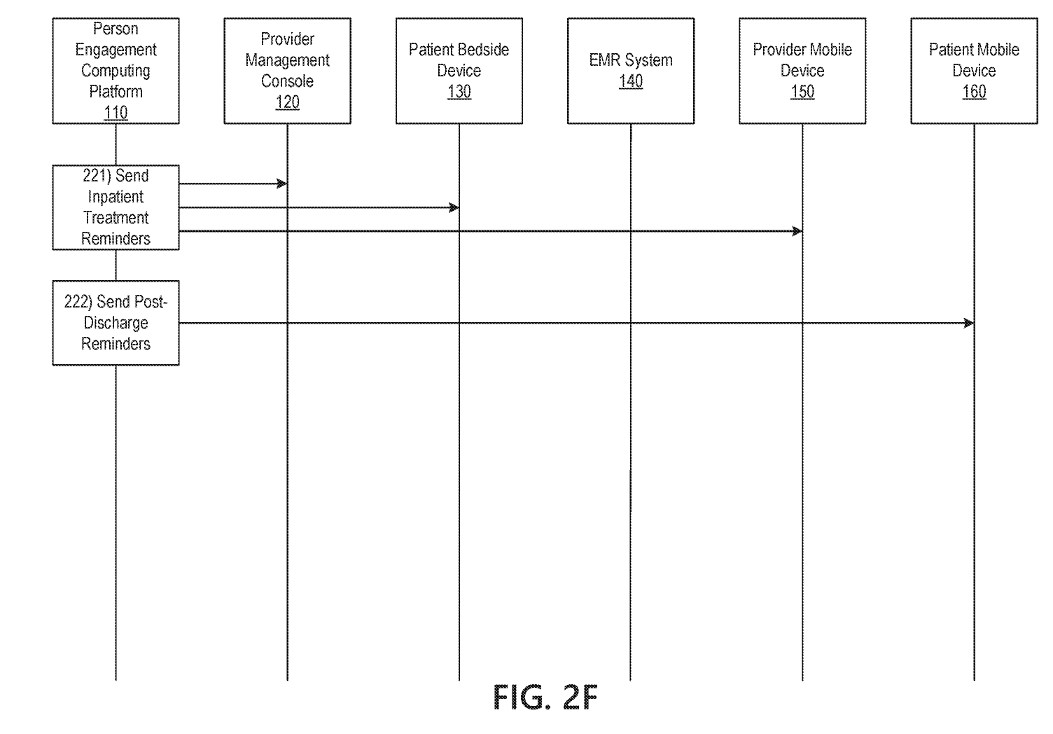

FIGS. 2A-2F depict an illustrative event sequence for implementing and using a data processing system in an inpatient context to provide automated personalized healthcare functions in accordance with one or more example embodiments. In these embodiments, a patient may be provided with a patient bedside device 130, which may communicate with the person engagement computing platform 110 in order to implement a process according to the illustrated event sequence.

Referring to FIG. 2A, at step 201, person engagement computing platform 110 receives patient information from provider management console 120 as part of a patient admission notification. For example, during a patient admissions procedure, clinicians and/or administrative staff may determine the patient's name and/or some other patient identifier, which may be entered into provider management console 120. The staff may additionally collect other patient information such as one or more symptoms, a reason for a visit, a type of admission (e.g., an inpatient admission), contact information, and other such patient information, which may be entered into provider management console 120 and/or determined or retrieved from other systems by provider management console 120. The provider management console 120 may communicate some or all of the patient information to person engagement computing platform 110 as part of a patient admission notification.

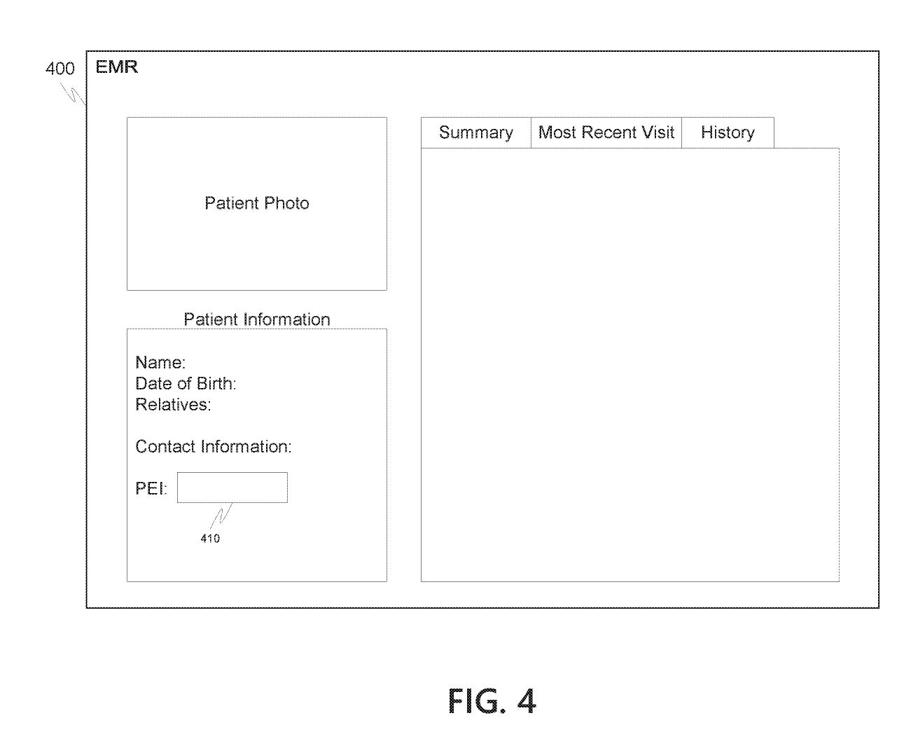

At step 202, person engagement computing platform 110 may send a request that causes the EMR system 140 to retrieve a record corresponding to the patient identified by the patient information. The record may contain additional information about the patient from previous visits to the same medical facility or to another medical facility. FIG. 4 illustrates an example patient record 400 containing information such as a patient photo, patient information including a name, date of birth, relatives, and contact information. The record may also include symptoms, diagnoses, procedures, treatments, and other such information about current and past medical issues for the patient. The record and/or patient information contained in the record may be retrieved by EMR system 140 and communicated to person engagement computing platform 110, as illustrated.

In some embodiments, the patient record 400 may include a PEI field 410 for storing a PEI score in the patient record 400. In some instances, at step 202, the patient record 400 may already store a PEI score determined at a previous time (e.g., during a previous visit to a same or different medical facility). In these instances, the EMR system 140 may retrieve the stored PEI score 410 and transmit it to person engagement computing platform 110 at step 202.

At step 203, person engagement computing platform 110 and/or PEI calculation module 113a may estimate an initial person engagement index (PEI) score based on preliminary information. In some embodiments, if patient record 400 already contained a PEI score 410, person engagement computing platform 110 may use the stored PEI score 410 as the initial PEI score. An estimated initial PEI score may, in some embodiments, be determined based on one or more items of patient information obtained in steps 201 and/or 202, such as demographic information, a preferred contact method, a number of relatives, and other such patient information. For example, person engagement computing platform 110 may compare the patient demographic information to demographic information and associated PEI scores for other patients in order to estimate the patient's initial PEI score. Accordingly, demographic information and PEI scores for other patients may, in some embodiments, be stored in memory 112 of person engagement computing platform 110.

In some embodiments, person engagement computing platform 110 and/or PEI calculation module 113a may implement statistical and/or machine learning techniques to train one or more models to estimate an initial PEI score based on demographic information and PEI information for other patients. For example, person engagement computing platform 110 may train a regression model to estimate an initial PEI score based on patient demographic information using training data, and such training data may include corresponding demographic information and PEI scores for other patients. Other such techniques and/or types of models may be used by person engagement computing platform 110 to estimate the initial PEI score.

As further discussed below, the PEI score may be determined (e.g., by person engagement computing platform 110) based on several PEI factor sub-scores indicating, for example, a technology use in healthcare factor of the PEI score. The PEI score may be a weighted average of the PEI factor sub-scores. Other patient information, such as preferred contact method, may affect a particular PEI factor sub-score (and correspondingly the PEI score). For example, a preferred contact method of “text message” or “email” may indicate a relatively high technology use in healthcare sub-score as compared to a “telephone” preferred contact method. Similarly, other patient information, such as a number of relatives, may indicate a relatively higher psychosocial support sub-score as compared to fewer relatives. Accordingly, the initial PEI score may be based on various types of patient information.

In some embodiments, person engagement computing platform 110 and/or PEI calculation module 113a may implement statistical and/or machine learning techniques to train one or more models to estimate an initial PEI score based on such additional information (e.g., a number of relatives, a preferred contact method, and the like) and PEI information for other patients. For example, person engagement computing platform 110 may train a regression model to estimate a PEI score based on such additional information (together with or separately from demographic information) using training data comprising corresponding additional information (together with or separately from demographic information) and PEI scores for other patients. Other such techniques and/or types of models may be used (e.g., by person engagement computing platform 110) to estimate the initial PEI score.

After determining the initial PEI score, person engagement computing platform 110 may cause the initial PEI score to be displayed at one or more of provider management console 120, patient bedside device 130, provider mobile device 150, and patient mobile device 160.

At step 204, person engagement computing platform 110 may establish a communication session with patient bedside device 130. In some embodiments, patient information (e.g., as received at step 201 or at another time) may include an indicator of the patient bedside device 130 associated with the patient. Accordingly, person engagement computing platform 110 may send (e.g., via communication interface 114) a request to the indicated patient bedside device 130, which may reply in order to establish the session, as illustrated. In some embodiments, the patient bedside device 130 may send a request to establish a communication session to the person engagement computing platform 110. Such a request received from patient bedside device 130 may include a patient identifier associated with the patient so that person engagement computing platform 110 may associate the session with the patient. The person engagement computing platform 110 may then reply to the request from patient bedside device 130 in order to establish the session. In some instances, person engagement computing platform 110 may associate the established session with information about the patient (e.g., the initial PEI score determined in step 203) and, responsive to initiating the session, continue the process at step 205.

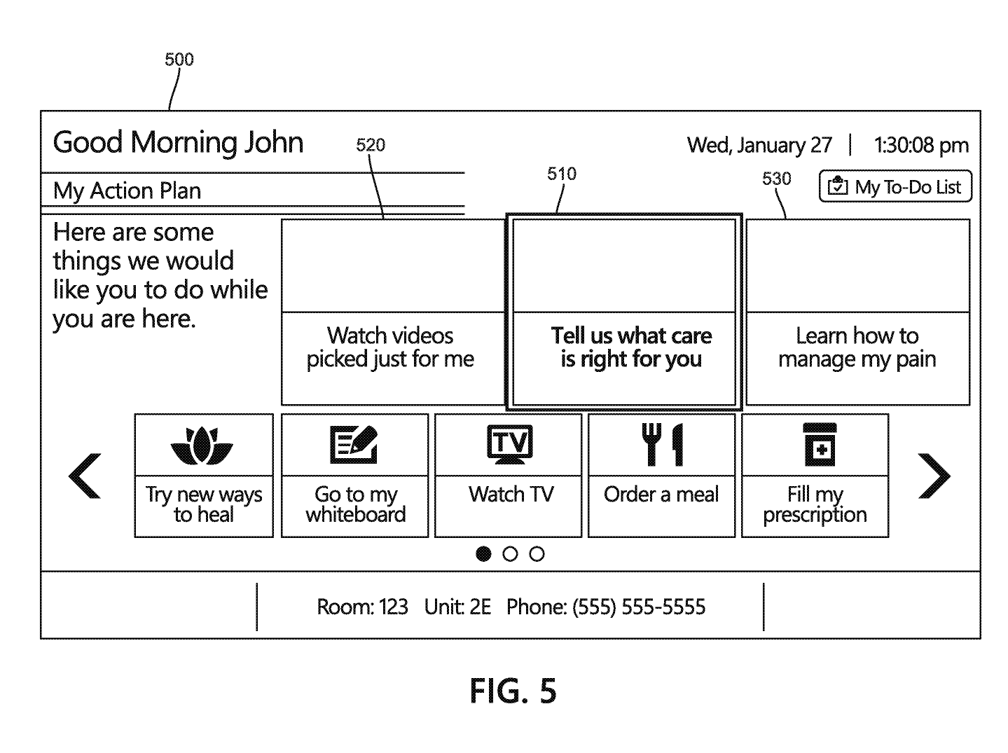

Referring to FIG. 2B, at step 205, person engagement computing platform 110 and/or portal generation module 113b may generate a patient portal and deliver the patient portal to the patient bedside device 130. The patient portal may be composed of and/or include one or more user interfaces that may enable and disable options for providing entertainment, educational, and medical information to patients. FIG. 5 illustrates an example patient portal 500. As illustrated, person engagement computing platform 110 and/or patient bedside device 130 may customize the patient portal for a particular patient. In some embodiments, the patient portal 500 may be customized in accordance with the initial PEI score. The patient bedside device 130 may display the patient portal and receive inputs from the patient in order to create an interactive experience.

Patients and other healthcare consumers, especially in an inpatient context, may often experience feelings of boredom, confusion, isolation, or other negative feelings that may leave the patient unsatisfied with their healthcare. Improving a patient's experience during an inpatient stay results in greater satisfaction and better healthcare outcomes. The patient portal (which may, e.g., be provided by person engagement computing platform 110) may improve a patient's experience by providing entertainment and educational content customized for each patient, based on the patient's health issues and the patient's engagement in healthcare, as measured by the PEI score.

Accordingly, the patient portal (which may, e.g., be provided by person engagement computing platform 110) may enable a link 510 for accessing a survey that may be used to determine a PEI score for the patient. In some situations, the link 510 may be featured prominently within the patient portal 500. For example, the patient portal 500 may enable a dynamic list of links to entertainment and medical content, such as a list of videos chosen for the patient, and a pain management plan tailored to the patient. Such a dynamic list may feature the link 510 when a patient has not yet completed some or all of a survey for determining a PEI score.

Accordingly, at step 206, the person engagement computing platform 110 and/or portal generation module 113b may be configured to enable a patient survey. In some embodiments, the survey is provided via the patient bedside device 130. For example, the patient bedside device 130 may be configured to access, upon a patient selecting the link 510, a web page or other user interface generated by person engagement computing platform 110 (and/or another device, such as a third party survey tool) for interacting with the survey. Accordingly, in some embodiments, the survey may be rendered within a browser executed by the patient bedside device 130. In some embodiments, the survey may be rendered in some other application executed by the patient bedside device 130. In some instances, if the patient does not select link 510, the person engagement computing platform 110 may proactively send a notification to the patient bedside device 130 and/or the patient mobile device 160, and the notification may prompt the patient to a take the survey. In some embodiments, person engagement computing platform 110 may select the mechanism for delivering the notification based on the initial PEI score. For example, if the initial PEI score indicates a relatively low technology use in healthcare sub-score, the notification may be delivered via a telephone call, whereas if the initial PEI score indicates a relatively high technology use in healthcare sub-score, the notification may be delivered via text message, via email, as a notification delivered via an application of the patient bedside device 130, or via some other mechanism. Accordingly, a PEI score may be used (e.g., by person engagement computing platform 110) to determine a preferred contact method and deliver notifications accordingly. Such a preferred contact method may be stored in a record 400 and/or displayed for the healthcare provider (e.g., at provider management console 120).

In some embodiments, the survey may be delivered via an application on a provider mobile device 150. For example, a provider mobile device 150 may execute an application for mobile patient rounding and data collection. Such an application may integrate with person engagement computing platform 110 to display a survey in addition to or in place of the survey delivered via patient portal 500. In some embodiments, a clinician or healthcare provider may administer the survey via provider mobile device 150 based on a patient's ability and/or willingness take a survey via the patient bedside device 130. Accordingly, person engagement computing platform 110 may deliver the survey via the provider mobile device 150 depending on a condition of the patient. In some instances, person engagement computing platform 110 may deliver the survey via the patient mobile device 160 when the patient is associated with a low initial PEI score.

In some embodiments, person engagement computing platform 110 may enable an alternate mechanism for delivering the survey (and/or disable the survey provided via the patient portal 500). For example, the survey may be provided via an automated telephone system. In some embodiments, the person engagement computing platform 110 may deliver the survey via an automated telephone system if a patient is associated with a relatively low PEI score and/or technology use in healthcare sub-score of the PEI score.

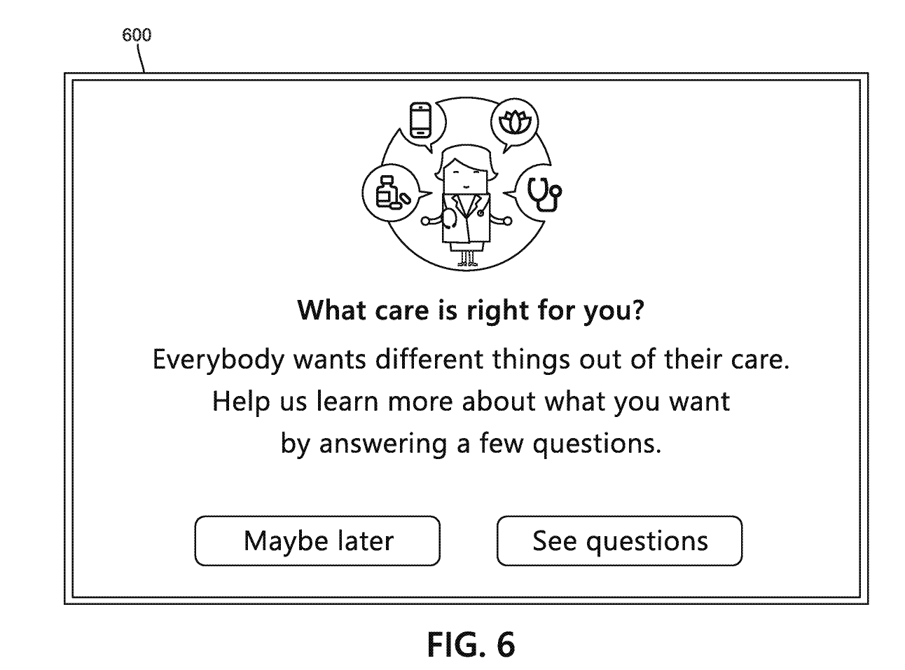

FIG. 6 illustrates an example prompt 600 that may be rendered on patient bedside device 130 (and/or another device such as patient mobile device 160) in response to receiving a notification from person engagement computing platform 110. The person engagement computing platform 110 and/or patient bedside device 130 may cause the display of the prompt 600 at patient bedside device 130 when a patient has not yet started the survey. The prompt 600 may contain links or functions for beginning the survey and/or postponing the survey. In some embodiments, when a patient still has not taken the survey after patient bedside device 130 has displayed the prompt 600, person engagement computing platform 110 and/or patient bedside device 130 may cause patient bedside device 130 to again display prompt 600. In some embodiments, person engagement computing platform 110 and/or patient bedside device 130 may cause the prompt 600 to display repeatedly at a frequency determined (e.g., by person engagement computing platform 110) based on the initial PEI score. For example, a patient with an initial PEI score indicating a relatively low engagement in healthcare sub-score (e.g., less than a threshold score or sub-score) may be prompted less frequently (e.g., in order to avoid bothering the patient) than a patient with a higher engagement in healthcare sub-score.

In some embodiments, person engagement computing platform 110 may send a notification to a provider mobile device 150 associated with a clinician or administrative staff based on the initial PEI score. For example, when an initial PEI score indicates a relatively low technology use in healthcare sub-score (e.g., less than a threshold score or sub-score), a notification may be sent to the provider mobile device 150 indicating that the patient may need assistance in accessing and completing the survey.

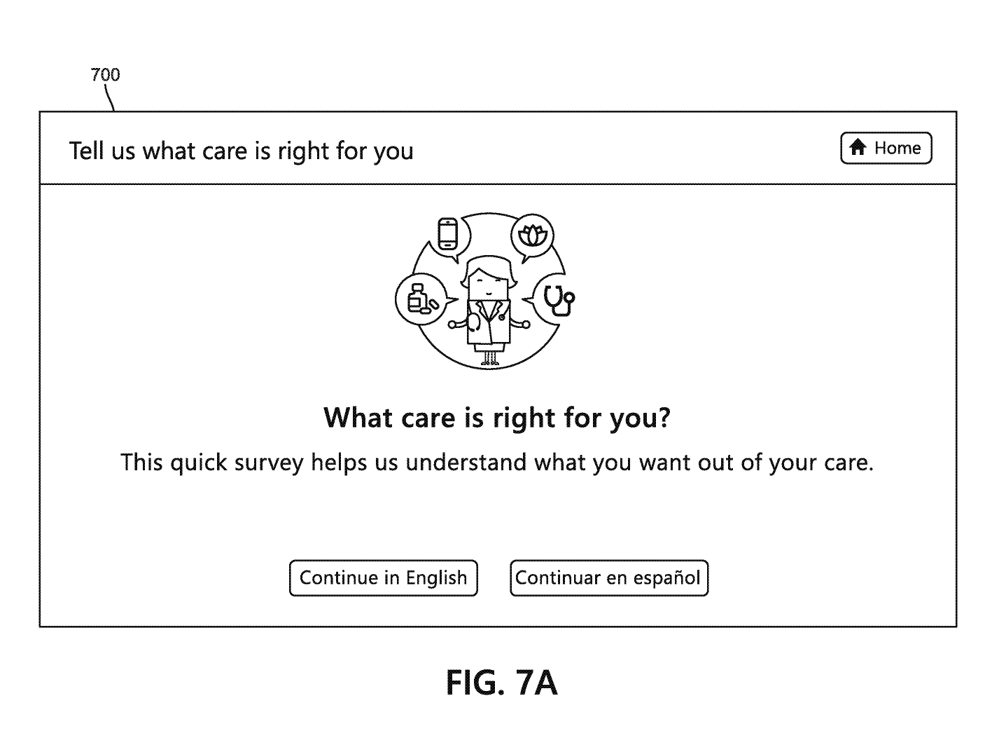

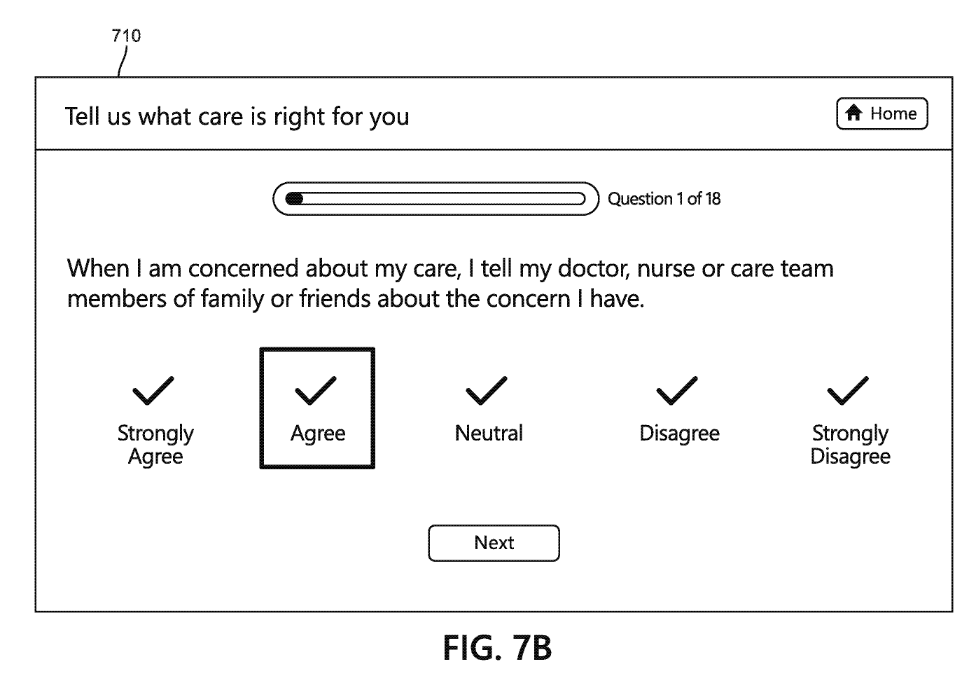

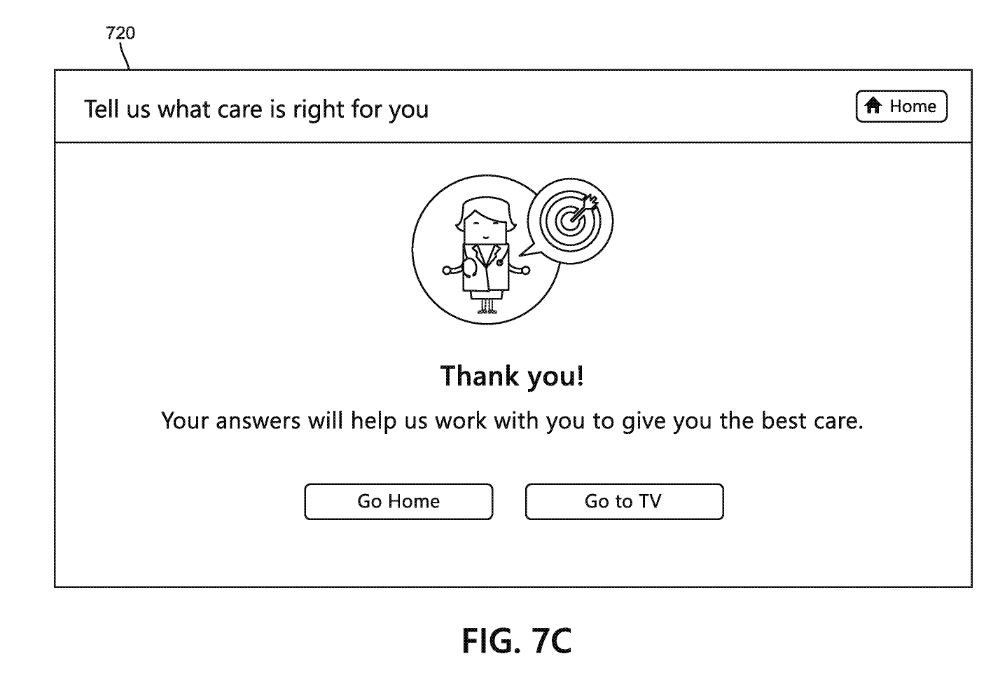

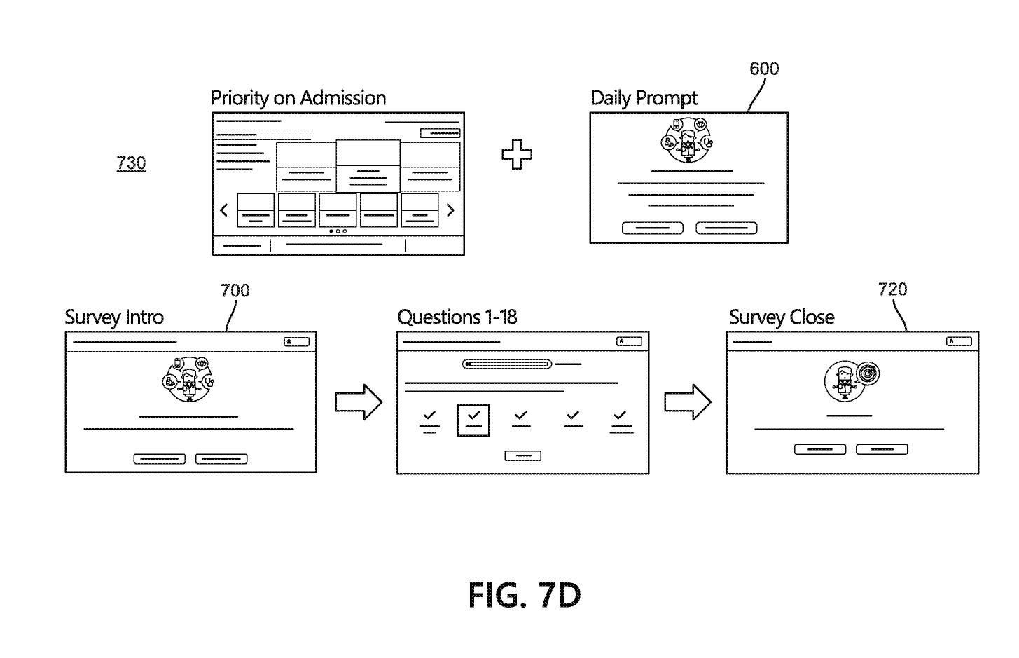

FIG. 7A illustrates an example user interface 700 that patient bedside device 130 may present upon receiving an indication that the patient wishes to begin the survey. As illustrated, the user interface 700 may provide a function for selecting a language for taking the survey. FIG. 7B illustrates an example user interface 710 for presenting one of the questions of the survey. The patient bedside device 130 may present a series of questions in turn and receive corresponding answers. As illustrated, the questions may be presented and/or the answers may be received according to a five-point Likert scale. However, other rating scales may be used in other embodiments. FIG. 7C illustrates a user interface 720 that patient bedside device 130 may present upon receiving answers to all of the questions of the survey. As illustrated, the user interface 720 may include links to access other functions of the patient bedside device 130, such as entertainment functions or other user interfaces.

FIG. 7D illustrates a first example workflow for surveying a patient according to step 206. This workflow and/or the other example workflows discussed herein may be directed by person engagement computing platform 110 and/or performed according to user interfaces generated by and/or otherwise provided by person engagement computing platform 110. As illustrated, upon admission, a patient bedside device 130 (and/or another device such as patient mobile device 160) may present a patient portal featuring a link 510 for taking the survey. Additionally or alternatively, the patient bedside device 130 may regularly display a prompt to take the survey when the patient has not yet finished the survey. When the patient accesses the survey, the patient bedside device 130 may display the user interface 700, followed by the user interface 710 (e.g., for each question of the survey), followed by the user interface 720.

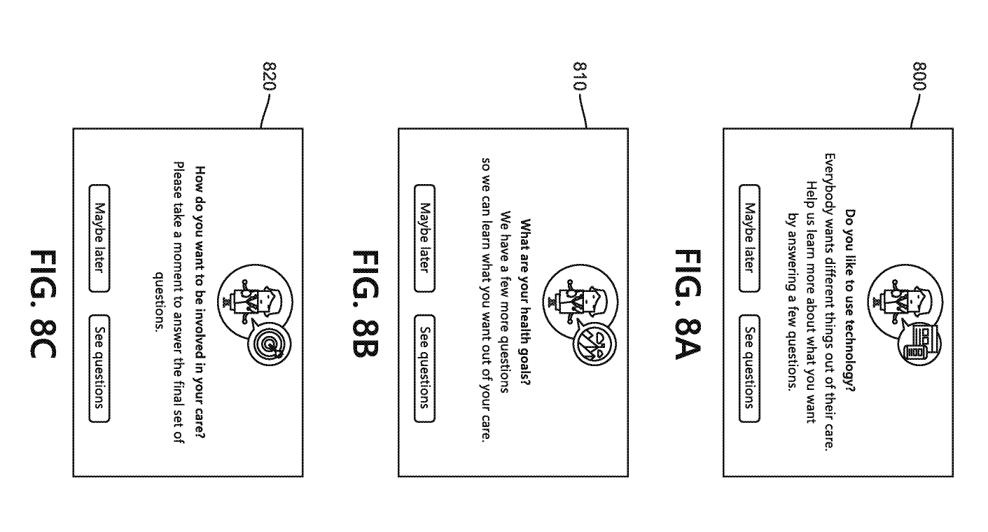

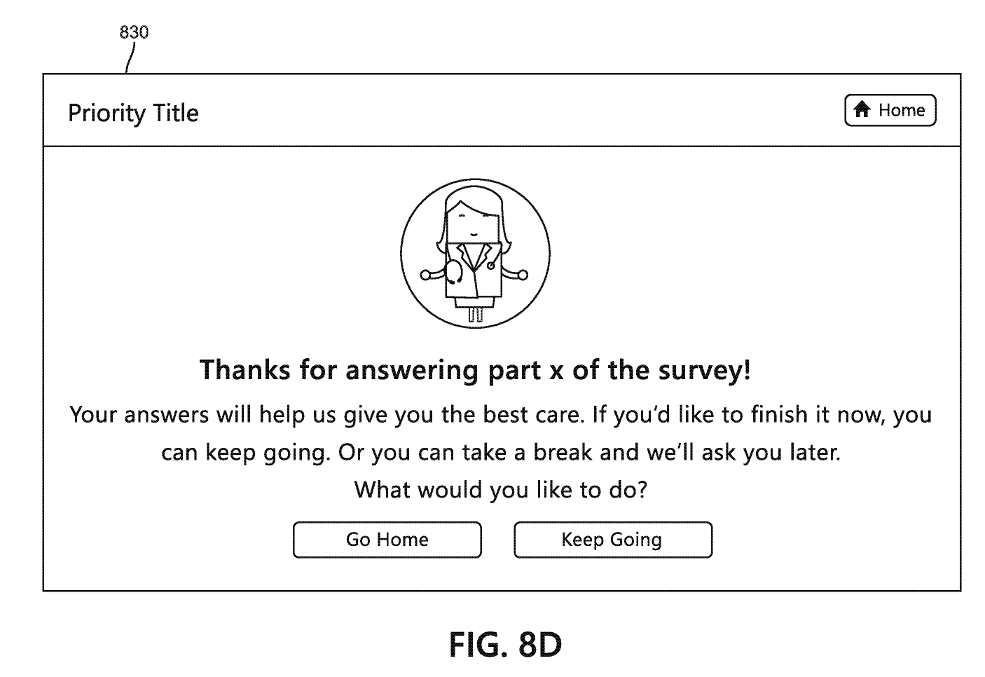

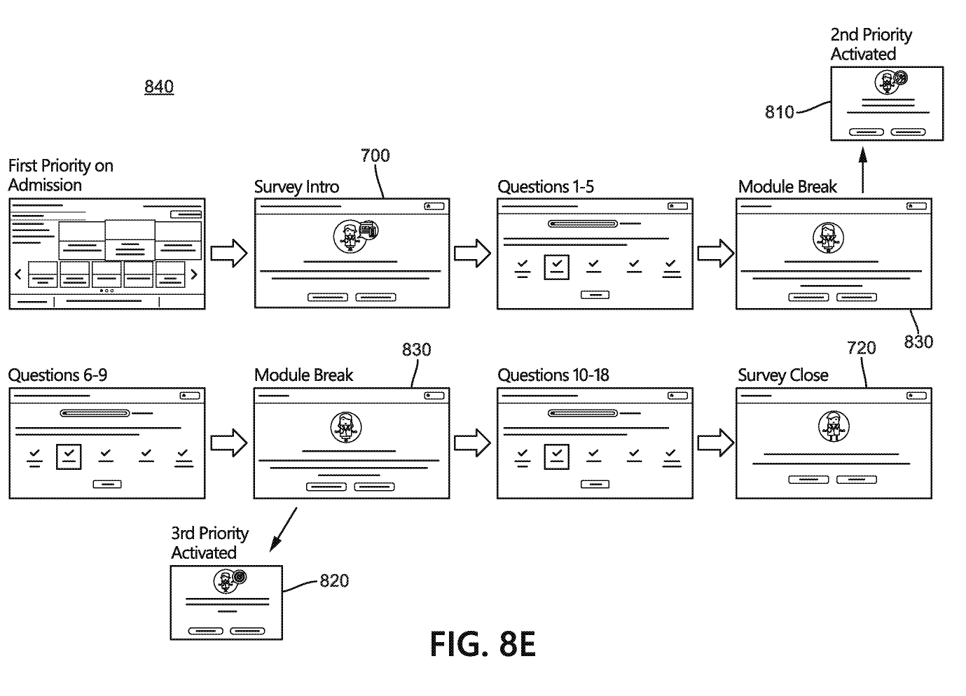

In some embodiments, the survey may be divided into components or modules, which patient bedside device 130 may enable separately (e.g., at different times). Accordingly, patient bedside device 130 may display one or more prompts to take different survey modules. FIGS. 8A, 8B, and 8C illustrate example prompts 800, 810, and 820, respectively. A first example prompt 800 may prompt a patient to take a first survey module pertaining to a technology use in healthcare sub-score of the PEI score. A second example prompt 810 may prompt a patient to take a second survey module pertaining to one or more additional sub-scores of the PEI score. A third example prompt 820 may prompt a patient to take a third survey module pertaining to one or more other sub-scores of the PEI score.

The person engagement computing platform 110 and/or patient bedside device 130 may cause the display of such prompts 800, 810, 820 at patient bedside device 130 when a patient has not yet started the corresponding survey module. The prompts 800, 810, 820 may contain links or functions enabling access to the survey module and/or postponing the survey module. In some embodiments, when a patient still has not taken a survey module after patient bedside device 130 has displayed the corresponding prompt 800, 810, 820, person engagement computing platform 110 and/or patient bedside device 130 may cause patient bedside device 130 to again display the corresponding prompt 800, 810, 820.

In some embodiments, after receiving answers to a first survey module corresponding to a technology use in healthcare sub-score of the PEI score, person engagement computing platform 110 may calculate a partial PEI score and/or a technology use in healthcare sub-score of the PEI score. Based on the partial PEI score and/or PEI factor sub-score, the person engagement computing platform 110 may determine an alternative method for completing the surveying and/or prompting the patient to complete the survey. For example, if the partial PEI score and/or technology use in healthcare sub-score indicates a relatively low use of technology in healthcare (e.g., a score or sub-score falling below a threshold), the person engagement computing platform 110 may switch to an automated telephone system to deliver the remainder of the survey and/or the prompts to complete the survey.

FIG. 8D illustrates an example user interface 830 that patient bedside device 130 may display when a user completes a survey module displayed on patient bedside device 130. The user interface 830 may include links and/or functions for continuing on to the next survey module and for postponing the next survey module. FIG. 8E illustrates a second example workflow 840 for surveying the patient according to step 206. As illustrated, a patient may access the first survey module by selecting link 510 of user interface 500. After finishing the first survey module, the patient may continue to the second survey module or postpone the second survey module. If the user postpones the second survey module, person engagement computing platform 110 and/or patient bedside device 130 may cause patient bedside device 130 to enable a prompt 810 that provides access to the second survey module. Additionally or alternatively, link 510 of user interface 500 may provide access to the second survey module after the first survey module is completed. Similarly, after finishing the second survey module, the patient may continue to the third survey module or postpone the third survey module. If the patient postpones the third survey module, person engagement computing platform 110 and/or patient bedside device 130 may cause patient bedside device 130 to enable a prompt 820 that provides access to the third survey module. Additionally or alternatively, link 510 of user interface 500 may provide access to the third survey module after the second survey module is completed. In some embodiments, the survey may be divided into more or fewer than three survey modules. For example, in some embodiments, the survey may be divided into four survey modules, each survey module corresponding to one of the sub-scores of the PEI score.

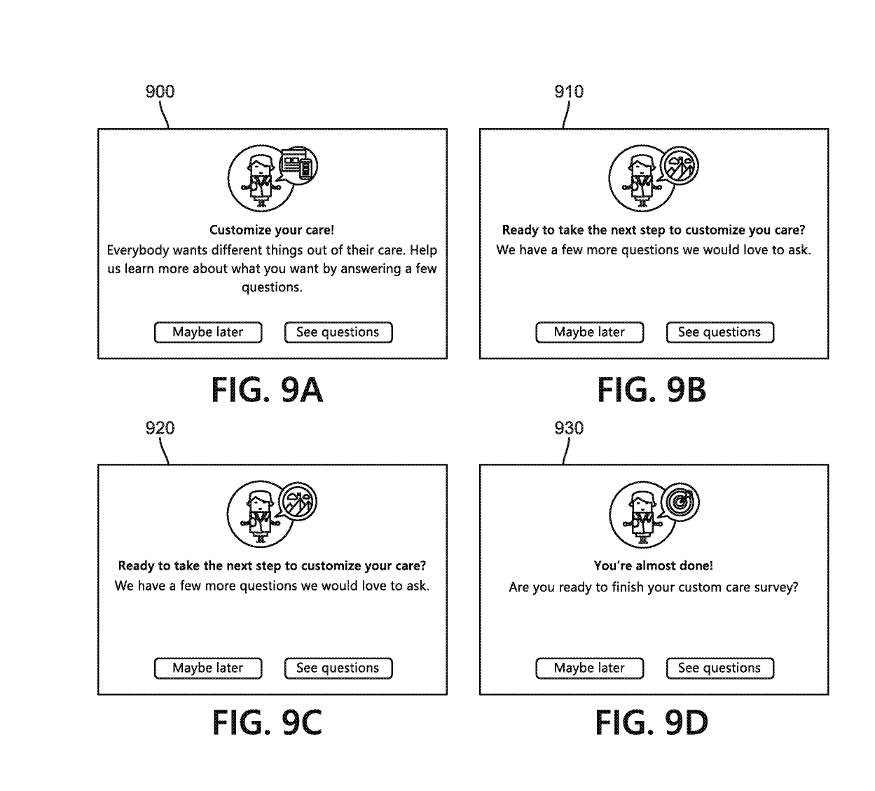

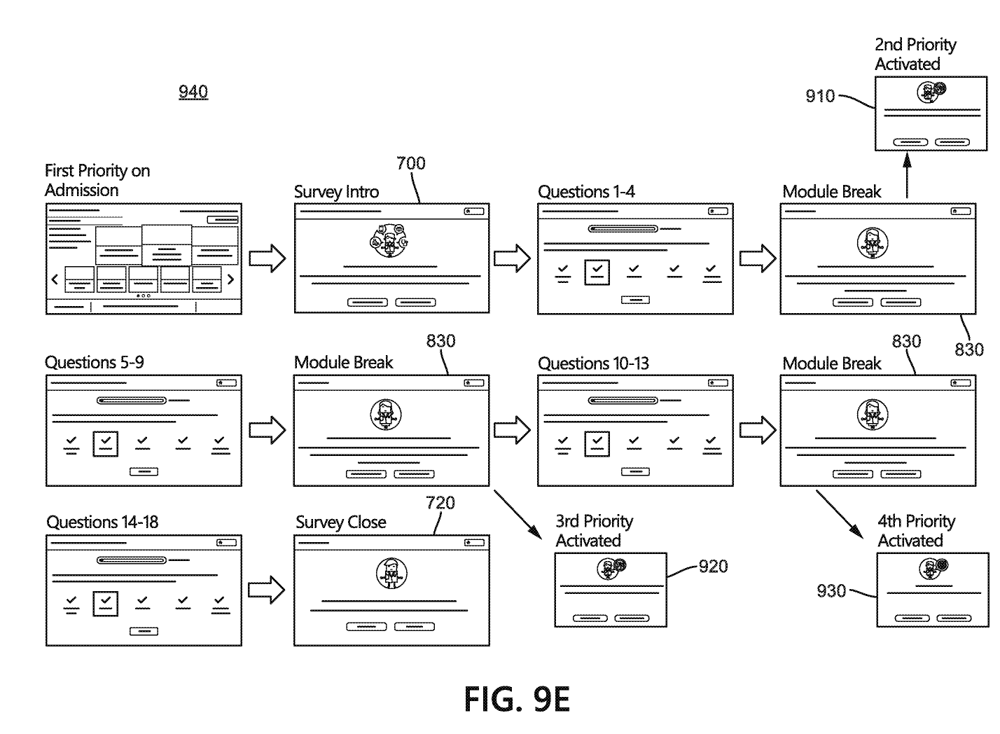

In some embodiments, each survey module may include one or more questions associated with each PEI factor sub-score of the PEI score. In these embodiments, person engagement computing platform 110 may generate a partial PEI factor sub-score after the patient completes each survey module. The person engagement computing platform 110 may further generate a partial PEI score based on the partial PEI factor sub-scores. Accordingly, patient bedside device 130 may enable one or more prompts for different survey modules. FIGS. 9A, 9B, 9C, and 9D illustrate example prompts 900, 910, 920, and 930, respectively. A first example prompt 900 may prompt a patient to take a first survey module, a second example prompt 910 may prompt the patient to take a second survey module, a third example prompt 920 may prompt the patient to take a third survey module, and a fourth example prompt 930 may prompt the patient to take a fourth survey module.

The person engagement computing platform 110 and/or patient bedside device 130 may cause the display of such prompts 900, 910, 920, 930 at patient bedside device 130 when a patient has not yet started the corresponding survey module. The prompts 900, 910, 920, 930 may contain links or functions for beginning the survey module and/or postponing the survey module. In some embodiments, when a patient still has not taken a survey module after patient bedside device 130 has displayed the corresponding prompt 900, 910, 920, 930, person engagement computing platform 110 and/or patient bedside device 130 may cause patient bedside device 130 to again display the corresponding prompt 900, 910, 920, 930.

FIG. 9E illustrates a third example workflow 940 for surveying the patient according to step 206. As illustrated, a patient may access the first survey module by selecting link 510 of user interface 500. After finishing the first survey module, the patient may continue to the second survey module or postpone the second survey module. If the patient postpones the second survey module, person engagement computing platform 110 and/or patient bedside device 130 may cause patient bedside device 130 to display a prompt 910 that provides access to the second survey module. Additionally or alternatively, link 510 of user interface 500 may provide access to the second survey module after the first survey module is completed. Similarly, after finishing the second survey module, the patient may continue to the third survey module or postpone the third survey module. If the patient postpones the third survey module, person engagement computing platform 110 and/or patient bedside device 130 may cause patient bedside device 130 to display a prompt 920 that provides access to the third survey module. Additionally or alternatively, link 510 of user interface 500 may provide access to the third survey module after the second survey module is completed. Finally, after finishing the third survey module, the patient may continue to the fourth survey module or postpone the fourth survey module. If the patient postpones the fourth survey module, person engagement computing platform 110 and/or patient bedside device 130 may cause patient bedside device 130 to display a prompt 930 that provides access to the fourth survey module. Additionally or alternatively, link 510 of user interface 500 may provide access to the fourth survey module after the third survey module is completed.

Returning to FIG. 2B, after the patient completes all or part of the survey at step 206, person engagement computing platform 110 and/or PEI calculation module 113a may calculate a PEI score at step 207 based on the survey answers provided. The person engagement computing platform 110 may determine the PEI score based on four PEI factors, including an engagement in healthcare factor, a technology use in healthcare factor, a proactive approach to healthcare factor, and a psychosocial support for healthcare factor. The person engagement computing platform 110 may assign a numeric score to each answer based on the selected answer option. For example, on the example user interface 710, a patient has selected an “Agree” answer, which may correspond to a numeric score of 4. In this example, the “Strongly Agree” answer may correspond to a numeric score of 3, the “Neutral” answer may correspond to a numeric score of 3, the “Disagree” answer may correspond to a numeric score of 2, and the “Strongly Disagree” answer may correspond to a numeric score of 1. However, other such correlations between answers and numeric scores may be used by person engagement computing platform 110 to determine the numeric score corresponding to each answer.

The person engagement computing platform 110 and/or PEI calculation module 113a may additionally determine which PEI factor and/or sub-factor each answer is associated with. In some embodiments, each question/answer may be associated with a single PEI factor and/or sub-factor. In other embodiments, some questions/answers may be associated with multiple PEI factors and/or sub-factors. The person engagement computing platform 110 may add up the numeric scores corresponding to a PEI factor to yield a raw factor score. Subsequently, the raw factor score may be normalized according to a scale (e.g., a 0-100 scale) to yield a PEI factor sub-score. The person engagement computing platform 110 may calculate the PEI score based on an average of the raw domain scores and/or PEI factor sub-scores. In some embodiments, the average may be a weighted average. The PEI score may be normalized according to a scale (e.g., a 0-100 scale).

In some embodiments, at step 207, person engagement computing platform 110 and/or PEI calculation module 113a may not have received all the answers (e.g., because a patient only completed a portion of the survey or less than all of the survey modules). In these instances, person engagement computing platform 110 may determine a partial PEI score based on the answers that were received. For example, when person engagement computing platform 110 has only received 3 answers associated with the engagement in healthcare PEI factor (e.g., out of a total of 9 on the survey), person engagement computing platform 110 may determine an average numeric score for the 3 answers that were received. Such an average numeric score may be normalized according to a scale (e.g., a 0-100) to determine a partial PEI sub-score. After determining multiple partial PEI sub-scores, person engagement computing platform 110 may determine a partial PEI score based on an average of the partial PEI sub-scores. In some embodiments, the averages may be weighted averages.

In some embodiments, person engagement computing platform 110 and/or PEI calculation module 113a may determine that insufficient answers have been received to generate a partial PEI sub-score. For example, person engagement computing platform 110 may have received no answers, or very few answers (e.g., less than 33% of the answers for a PEI factor), for a particular PEI factor. In these instances, person engagement computing platform 110 may use an estimated PEI factor sub-score (e.g., as determined at step 203) in place of determining a partial PEI factor sub-score for a PEI factor. Accordingly, person engagement computing platform 110 may determine the partial PEI score based in part on survey answers and based in part on demographic information or other information used to estimate a PEI factor sub-score.

After determining the PEI score and/or partial PEI score, person engagement computing platform 110 may cause the PEI score and/or partial PEI score to be displayed at one or more of provider management console 120, patient bedside device 130, provider mobile device 150, and patient mobile device 160.

At step 208, when a patient has not finished the survey, person engagement computing platform 110 and/or patient bedside device 130 may generate a notification that causes the patient bedside device 130 to enable a prompt to finish the survey. Such prompts may include prompts 800, 810, 820, 900, 910, 920, 930, or other such prompts. In some embodiments, the notification may instruct the patient bedside device 130 to overlay the prompt on the display of patient bedside device 130. For example, if a patient is watching television on the patient bedside device 130, the prompt may be overlaid on the television content. In some embodiments, the patient bedside device 130 may display a message that, when selected, causes display of one of the prompts 800, 810, 820, 900, 910, 920, or 930. The person engagement computing platform 110 and/or patient bedside device 130 may continually remind the patient to complete the survey by sending repeated notifications. In some embodiments, person engagement computing platform 110 and/or patient bedside device 130 sends the notifications at a frequency based on the partial PEI score. For example, a patient with a partial PEI score indicating a relatively low engagement in healthcare sub-score (e.g., less than a threshold score or sub-score) may be prompted less frequently (e.g., in order to avoid bothering the patient) than a patient with a higher engagement in healthcare sub-score. The person engagement computing platform 110 and/or patient bedside device 130 may determine which of prompts 800, 810, 820, 900, 910, 920, or 930 cause patient bedside device 130 to display based on the most recently-completed survey module, for instance, according to the workflows 840 or 940.

Referring to FIG. 2C, at step 209, person engagement computing platform 110 may receive additional answers to any questions that were not previously answered. At step 210, person engagement computing platform 110 may re-calculate a PEI score or partial PEI score based on the answers received in step 209. In some instances, steps 209-210 may occur repeatedly as a patient completes successive survey modules, until all survey modules are received. In some instances, a patient may never complete all the survey modules, and person engagement computing platform 110 may continue to use a partial PEI score for the remainder of the example event sequence. After determining the PEI score at step 210, person engagement computing platform 110 may cause the PEI score to be displayed at one or more of provider management console 120, patient bedside device 130, provider mobile device 150, and patient mobile device 160.

At step 211, person engagement computing platform 110 may optionally receive information about a patient treatment plan. The information about the patient treatment plan may be received from a provider management console 120, a provider mobile device 150, or some other computing system associated with a clinician or healthcare provider. The patient treatment plan may include information such as a patient rounding schedule (e.g., times when nurses check up on the patient), a medication schedule (e.g., times when the patient is scheduled to take particular medications), patient goals (e.g., the clinician may give the patient a goal of getting up and walking around the room three times a day to aid recovery), clinicians assigned to the patient (e.g., a doctor and/or nurse in charge of a patient), and other such treatment information.

The person engagement computing platform 110 and/or patient treatment plan module 113c may provide feedback and/or recommendations regarding aspects of the patient treatment plan based on the PEI score. Such feedback and/or recommendations may be transmitted to provider management console 120 in one or more notifications. At step 212, in some embodiments, person engagement computing platform 110 may provide a recommendation associated with a patient medication schedule based on the PEI score. In some instances, a low PEI score (or a low engagement in healthcare sub-score and/or a low proactive approach to healthcare sub-score) may tend to indicate that the patient may be less willing to follow and complete a patient medication schedule and/or may be less willing to take multiple medications. For such a patient, person engagement computing platform 110 may recommend modifying the patient medication schedule so that the patient takes multiple medications at the same time in order to reduce the number of times the patient must remember to take medication. For patients with higher PEI scores, the person engagement computing platform 110 may avoid recommending that a patient medication schedule should be modified so that the patient takes medications at the same time. Accordingly, person engagement computing platform 110 may recommend one or more modifications to the patient medication schedule based on the PEI score. Such recommendations may be provided to the provider management console 120 and/or other computing system associated with the healthcare provider.

In some embodiments, person engagement computing platform 110 and/or patient treatment plan module 113c may use one or more models (e.g., machine learning models, statistical models, and the like) to analyze the patient medication schedule with respect to the PEI score in order to generate one or more recommendations regarding the patient medication schedule. Such models may have been trained (e.g., by person engagement computing platform 110 or another computing system) based on historical data correlating PEI scores to patient medication schedule compliance. Such a trained model may output an effectiveness score for the patient medication schedule and/or one or more recommendations for improving the effectiveness score. In some embodiments, person engagement computing platform 110 may use one or more rules to generate recommendations. For example, a rule may indicate that for patients with a PEI score below a certain threshold, the patient medication schedule should not require the patient to take medications more than a certain number of times in a given time period (e.g., more than twice daily).

In addition to or as an alternative to providing recommendations about the patient medication schedule, the person engagement computing platform 110 and/or patient treatment plan module 113c may display an effectiveness score pertaining to the patient medication schedule. The person engagement computing platform 110 may generate such an effectiveness score based on the PEI score using trained models and/or rules. The person engagement computing platform 110 may cause the effectiveness score to be displayed on a device associated with a clinician or healthcare provider (e.g., the provider management console), and update the effectiveness score based on changes or modifications to the patient medication schedule received from the clinician or healthcare provider.

At step 213, person engagement computing platform 110 and/or patient treatment plan module 113c may determine a patient reminder schedule based on the PEI score. In some embodiments, a low PEI score (or a low engagement in healthcare sub-score and/or a low proactive approach to healthcare sub-score) may indicate that a patient needs more reminders in order to accomplish tasks such as taking medications, achieving patient goals, and performing other tasks. In contrast, a higher PEI score (or a higher engagement in healthcare sub-score and/or a higher proactive approach to healthcare sub-score) may indicate that the patient does not need frequent reminders in order to accomplish tasks. Accordingly, person engagement computing platform 110 may recommend providing more or fewer notifications for patient tasks such as taking medications, accomplishing goals, and other patient tasks based on the PEI score.

In some embodiments, person engagement computing platform 110 and/or patient treatment plan module 113c may use one or more models (e.g., machine learning models, statistical models, and the like) to analyze a patient reminder schedule with respect to the PEI score in order to generate one or more recommendations regarding the patient reminder schedule. Such models may have been trained (e.g., by person engagement computing platform 110 or another computing system) based on historical data correlating PEI scores to patient task compliance. Such a trained model may output an effectiveness score for the patient reminder schedule and/or one or more recommendations for improving the effectiveness score. In some embodiments, person engagement computing platform 110 may use one or more rules to generate recommendations. For example, a rule may indicate that for patients with a PEI score below a certain threshold, the patient reminder schedule should remind a patient to complete a task a certain number of times in a certain time period (e.g., at least once daily).

In addition to or as an alternative to providing recommendations about the patient reminder schedule, the person engagement computing platform 110 and/or patient treatment plan module 113c may display an effectiveness score pertaining to the patient reminder schedule. The person engagement computing platform 110 may generate such an effectiveness score based on the PEI score using trained models and/or rules. The person engagement computing platform 110 may cause the effectiveness score to be displayed on a device associated with a clinician or healthcare provider (e.g., the provider management console), and update the effectiveness score based on changes or modifications to the patient reminder schedule received from the clinician or healthcare provider.

At step 214, person engagement computing platform 110 and/or patient treatment plan module 113c may determine a patient rounding schedule based on the PEI score. In some embodiments, a low PEI score (or a low proactive approach to healthcare sub-score) may indicate that a patient needs more checkups. In contrast, a higher PEI score (or a higher proactive approach to healthcare sub-score) may indicate that the patient may be more likely to call for assistance when the patient has a problem or issue. Accordingly, person engagement computing platform 110 may recommend providing more or less frequent visits from nurses or other clinicians as part of a patient rounding schedule based on the PEI score.

In some embodiments, person engagement computing platform 110 and/or patient treatment plan module 113c may use one or more models (e.g., machine learning models, statistical models, and the like) to analyze a patient rounding schedule with respect to the PEI score in order to generate one or more recommendations regarding the patient reminder schedule. Such models may have been trained (e.g., by person engagement computing platform 110 or another computing system) based on historical data correlating PEI scores to patient satisfaction with patient rounding frequency, healthcare outcomes, or other such data. Such a trained model may output an effectiveness score for the patient rounding schedule and/or one or more recommendations for improving the effectiveness score. In some embodiments, person engagement computing platform 110 may use one or more rules to generate recommendations. For example, a rule may indicate that for patients with a PEI score below a certain threshold, the patient rounding schedule should include more frequent visits (e.g., hourly visits).

In addition to or as an alternative to providing recommendations about the patient rounding schedule, the person engagement computing platform 110 and/or patient treatment plan module 113c may display an effectiveness score pertaining to the patient rounding schedule. The person engagement computing platform 110 may generate such an effectiveness score based on the PEI score using trained models and/or rules. The person engagement computing platform 110 may cause the effectiveness score to be displayed on a device associated with a clinician or healthcare provider (e.g., the provider management console), and update the effectiveness score based on changes or modifications to the patient rounding schedule received from the clinician or healthcare provider.

At step 215, person engagement computing platform 110 and/or patient treatment plan module 113c may provide an overall treatment plan effectiveness rating. For example, person engagement computing platform 110 may calculate an average based on the effectiveness ratings for one or more of the patient medication schedule, the patient reminder schedule, and the patient rounding schedule. Accordingly, person engagement computing platform 110 may determine a personalized and comprehensive effectiveness rating of the treatment the patient will receive using the PEI score. The person engagement computing platform 110 may further cause the score to be displayed at a device associated with a clinician or healthcare provider (e.g., the provider management console 120).

Referring to FIG. 2E, at step 216, person engagement computing platform 110 and/or patient treatment plan module 113c may update any aspect of the patient treatment plan based on modification or updates received from a clinician or healthcare provider. If no such modifications or updates are received, person engagement computing platform 110 may confirm the treatment plan received at step 211.

At step 217, person engagement computing platform 110 may select one or more items of personalized educational content based on the patient information, the patient treatment plan, and the PEI score. The person engagement computing platform 110 may select educational content pertaining to one or more conditions, illnesses, symptoms, medications, treatments, or procedures associated with the patient. Such educational content may include videos, images, audio, and/or text stored in content database 113d.

In some embodiments, person engagement computing platform 110 may select one or more items of educational content from a content database 113d containing educational content based on matches between the one or more conditions, illnesses, symptoms, medications, treatments, or procedures associated with the patient and the educational content in the content database 113d. For example, the educational content in the content database 113d may be tagged with keywords, topics, or other metadata that may be compared (e.g., by person engagement computing platform 110) to the one or more conditions, illnesses, symptoms, medications, treatments, or procedures associated with the patient in order to determine a match and select the matched educational content. In some embodiments, person engagement computing platform 110 may implement or use a search engine to select educational content from the content database 113d using queries formulated based on the one or more conditions, illnesses, symptoms, medications, treatments, or procedures associated with the patient. In some embodiments, the matched content may be associated with a rank score (e.g., to yield an ordered list of educational content) based on how closely the content matches the one or more conditions, illnesses, symptoms, medications, treatments, or procedures associated with the patient.

In some embodiments, person engagement computing platform 110 may adjust the rank scores of the selected content based on the PEI score. For example, for patients with a lower PEI score, more engaging content such as videos, images, and audios may be preferred. Accordingly, person engagement computing platform 110 may reduce the rank score of less engaging content such as textual articles for patients with lower PEI scores. Similarly, person engagement computing platform 110 may increase a rank score for more engaging content for patients with lower PEI scores. For patients with higher PEI scores, person engagement computing platform 110 may increase a rank score of more detailed and/or more informative content. The person engagement computing platform 110 may store and execute one or more rules for increasing or decreasing the rank score of content based on the PEI score and based on the content type (e.g., video, images, text, etc.), the level of detail associated with the content (e.g., a content length, Flesch Reading Ease score associated with the content, or some other measure of the level of detail of the content), or other content categories or metrics. For example, person engagement computing platform 110 may store and execute a rule that decreases rank scores of items of educational content by a certain amount for patients with a PEI score above and/or below certain thresholds.

In some embodiments, education content in the content database 113d may be tagged with a target PEI score. For example, a very detailed article about advanced treatment options for a condition may be tagged with a relatively high target PEI score of 100, whereas a more engaging video explaining the basics of a condition may be associated with a relatively moderate target PEI score of 60. Based on the difference between the patient's PEI score and the target PEI score for an item of educational content, the rank score of an item may be adjusted so that educational content with a target PEI score close to the patient's PEI score may be ranked relatively more highly in comparison to educational content with a target PEI score having a large difference with the patient's PEI score.

Accordingly, person engagement computing platform 110 may determine a ranked list of educational content based on patient information, the patient treatment plan, and the PEI score at step 217. At step 218, person engagement computing platform 110 may generate one or more commands configured to update the patient portal and/or other aspects of the patient bedside device 130 based on the results of steps 216 and/or 217, and person engagement computing platform 110 may send the one or more commands to the patient bedside device 130. Turning to FIG. 5, the patient portal 500 may contain a dynamic listing or arrangement of links that are personalized for the patient. In some instances, the person engagement computing platform 110 may generate one or more commands that enable a link 520 for accessing one or more items of personalized educational content. In the illustrated example, the link 520 may enable access to video content selected according to step 217. In some embodiments, a link 520 may enable access other types of educational content together with or separately from video content. In some embodiments, the person engagement computing platform 110 may generate one or more commands that configure the patient portal 500 to include different links for accessing different types of personalized educational content. Upon receiving a selection of one of the links, the patient bedside device 130 may display a plurality of options to select one or more items of educational content. In some embodiments, the person engagement computing platform 110 may generate one or more commands that configure the patient bedside device 130 to display the plurality of options arranged according to a rank score associated with the respective educational content.

The person engagement computing platform 110, at step 218, may generate one or more commands that update the patient portal 500 to include patient treatment plan information. For example, the one or more commands may enable link 530 to provide access to a pain management feature of the patient treatment plan, which may include information from the patient medication schedule, as well as other patient treatment plan information, such as rounding schedule information indicating when a nurse will next visit, educational content about pain management, and other content related to pain management. Accordingly, person engagement computing platform 110 may configure patient bedside device 130 to display information about aspects of the personalized patient treatment plan together with and/or separately from one or more items of personalized educational content.

The person engagement computing platform 110 may generate one or more commands that configure other aspects of the patient portal 500 and/or patient bedside device 130. In some embodiments, person engagement computing platform 110 may generate one or more commands that configure a patient whiteboard (which may be selectable via patient portal 500 or otherwise may be displayed on patient bedside device 130) to display information from the personalized patient treatment plan. For example, person engagement computing platform 110 may generate one or more commands that configure the patient whiteboard to display a schedule indicating times of scheduled procedures, meal times, medication times, and other items scheduled for the patient according to the patient treatment plan. In some embodiments, the one or more commands may configure the patient whiteboard to display one or more patient goals. In some embodiments, the one or more commands may configure the patient whiteboard to provide forms for allowing the patient to communicate with one or more clinicians assigned to the patient. The person engagement computing platform 110 may generate one or more commands that configure the patient portal to display a link that causes the patient bedside device 130 to display the patient whiteboard. In some embodiments, the person engagement computing platform 110 may generate one or more commands that cause the patient bedside device 130 to enable or disable notifications related to a survey (and/or modules of the survey), a patient treatment plan, a patient rounding schedule, a patient medication schedule, and/or a patient reminder schedule. In some embodiments, the person engagement computing platform 110 may generate one or more commands configured to cause the patient bedside device 130 to display a PEI score. Accordingly, the person engagement computing platform 110 may generate one or more configuration commands for the patient bedside device 130 based on the PEI score associated with the patient. Subsequently, the person engagement computing platform 110 may send the one or more configuration commands to the patient bedside device 130. The patient bedside device 130 may, based on the one or more configuration commands, selectively enable or disable one or more functions of the patient bedside device 130 based on the PEI score associated with the patient.

At step 219, person engagement computing platform 110 may receive content interaction information from patient bedside device 130. The patient bedside device 130 may monitor a patient's interactions with the personalized educational content accessible via the patient portal 500 and generate the content interaction information based on the monitored interactions. For example, the patient bedside device 130 may track which educational content items the patient selects and/or an order of such selections, how long the patient spends viewing each educational content item, and the like. In some embodiments, patient bedside device 130 may track start and end times associated with video content. For example, patient bedside device 130 may determine which portions of a video content item were viewed, whether a patient skipped certain portions, whether a patient ended a video early, and the like by storing timestamps indicating that a patient began watching a video at a certain point (e.g., by pressing play or by skipping to the point) and stopped the video at another point (e.g., by skipping away at the point, by stopping at the point, etc.). Accordingly, patient bedside device 130 may track one or more selections and/or start and end timestamps for video educational content. Any and/or all of the information tracked by patient bedside device 130 may be included in the content interaction information. In addition, the patient bedside device 130 may periodically transmit the content interaction information to person engagement computing platform 110.

At step 220, based on the received content interaction information, person engagement computing platform 110 and/or PEI calculation module 113a may optionally update the PEI score. In some embodiments, person engagement computing platform 110 may raise a PEI score based on content interaction information showing a high engagement with the educational content. For example, based on a patient with a relatively low PEI score viewing detailed and/or advanced educational content, the patient's PEI score may be adjusted upwards. In some embodiments, person engagement computing platform 110 may implement rules to adjust the PEI score based on PEI score thresholds, a content type, a level of detail associated with the content, and/or other educational content scores or metrics. An example rule may indicate that a PEI score of less than 60 may be increased by 5 points if the patient spends at least 10 minutes viewing video educational content, and may be increased by 10 points if the patient spends at least 20 minutes viewing textual educational content with a high level of detail. As another example, a rule may provide a formula for adjusting a PEI score upwards in proportion to time spent viewing educational content and the average level of detail associated with the viewed educational content. In some embodiments, person engagement computing platform 110 may use one or more rules for lowering a PEI score based on content interaction information showing low engagement with educational content. For example, if a patient fails to complete viewing one or more items of video educational content, a PEI score may be lowered. The person engagement computing platform 110 may execute such rules and update the PEI score accordingly. After updating and/or adjusting the PEI score, person engagement computing platform 110 may cause the updated PEI score to be displayed at one or more of provider management console 120, patient bedside device 130, provider mobile device 150, and patient mobile device 160.

In some embodiments, person engagement computing platform 110 and/or PEI calculation module 113a may adjust the PEI score based on a target PEI score associated with educational content indicated in the content interaction information. For example, if a patient with a PEI score of 70 views an item of educational content with a target PEI score of 80, the patient's PEI score may be adjusted upwards. In some embodiments, person engagement computing platform 110 may calculate the average target PEI score of several items of educational content, and adjust a PEI score (e.g., upwards) in proportion to the difference between the average target PEI score and patient's PEI score, such that viewing educational content with a higher target PEI score may cause the patient's PEI score to be raised by a relatively higher amount.

Referring to FIG. 2F, at step 221, person engagement computing platform 110 may send one or more reminders to devices associated with clinicians or healthcare providers, such as provider management console 120 and provider mobile device 150 and/or to the patient bedside device 130, in accordance with the patient treatment plan. In some instances, the one or more reminders may remind the patient, clinician, and/or healthcare provider about scheduled items, such as a scheduled rounding or a scheduled dose of medication for the patient. In some instances, the one or more reminders may remind the patient, clinician, and/or healthcare provider about one or more patient goals. In some embodiments, reminders may be sent to one or more relatives of the patient (e.g., as determined from the EMR system 140 at step 202) in addition to or as an alternative to the patient, clinician, and/or healthcare provider. In some embodiments, person engagement computing platform 110 may send reminders to relatives of the patient when the patient is associated with a low PEI score (and/or a low engagement in healthcare and/or proactive approach to healthcare sub-score).

At step 222, person engagement computing platform 110 may send one or more post-discharge reminders to a device associated with the patient (e.g., reminders to take medications, reminders to schedule and/or attend follow-up procedures, and the like), such as patient mobile device 160. In some embodiments, the post-discharge reminders may be sent to one or more relatives of the patient (e.g., as determined from the EMR system 140 in step 202) in addition to or as an alternative to the patient. In some embodiments, person engagement computing platform 110 may send reminders to relatives of the patient when the patient is associated with a low PEI score (and/or a low engagement in healthcare and/or proactive approach to healthcare sub-score).

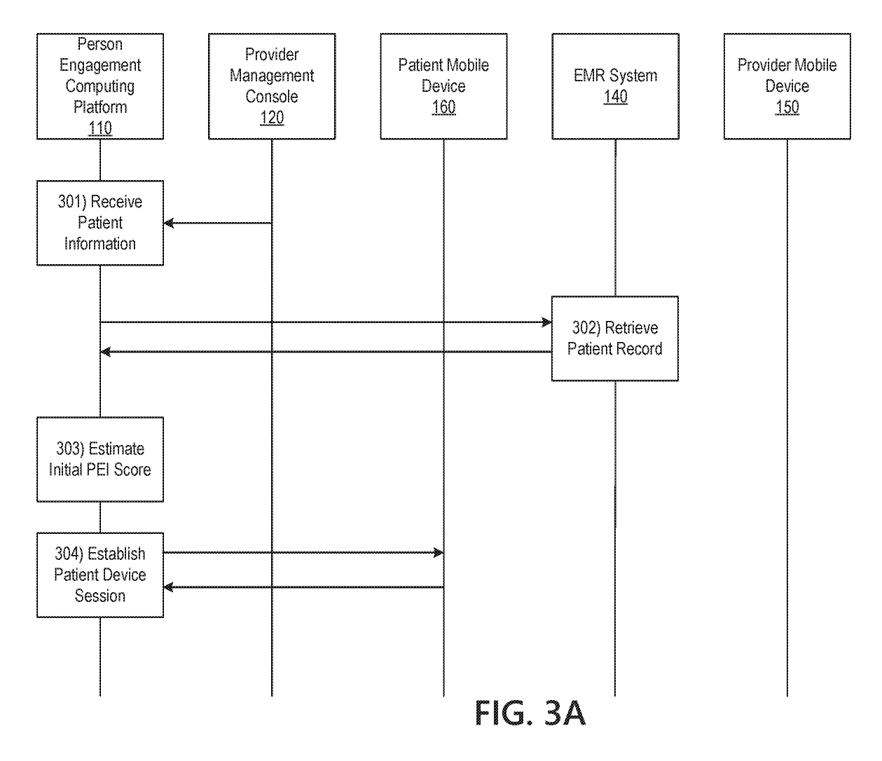

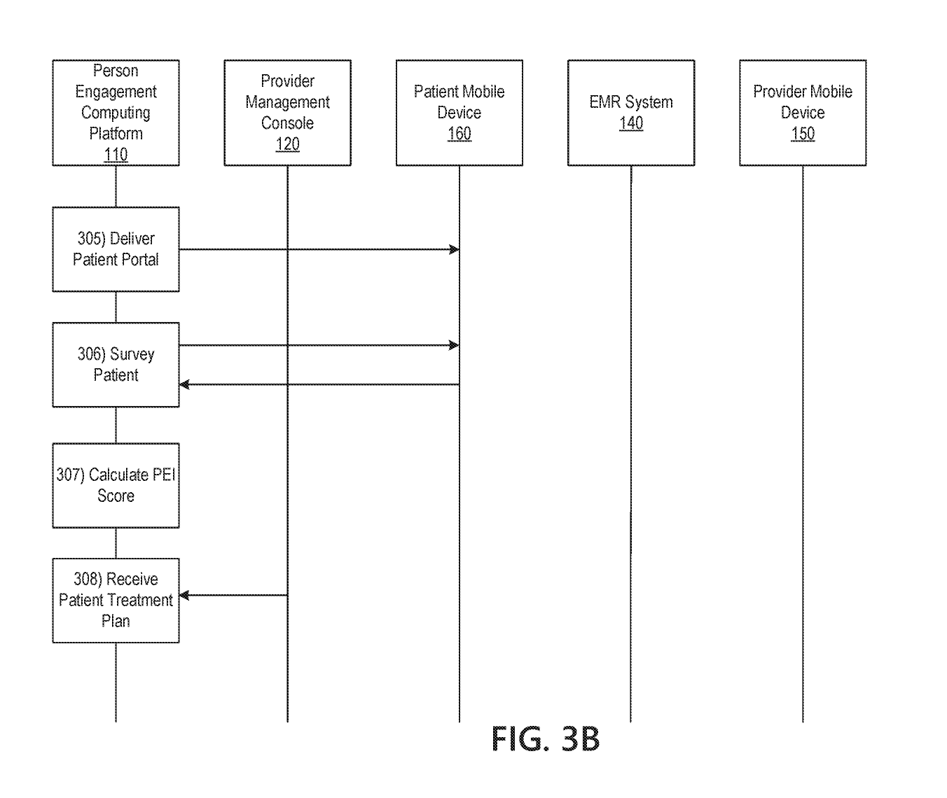

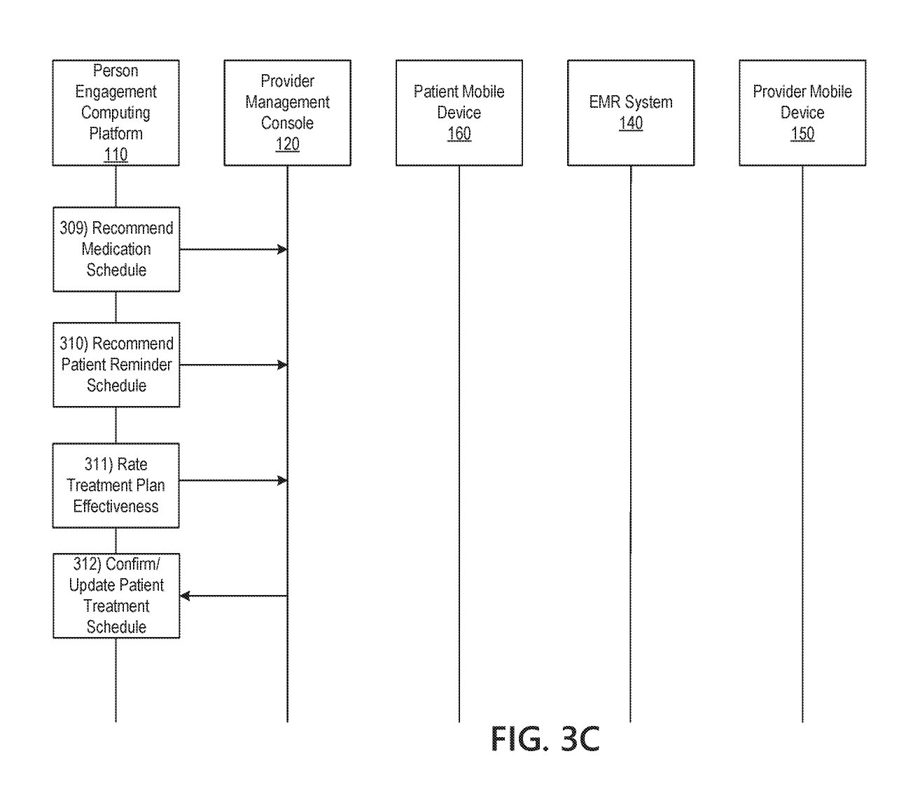

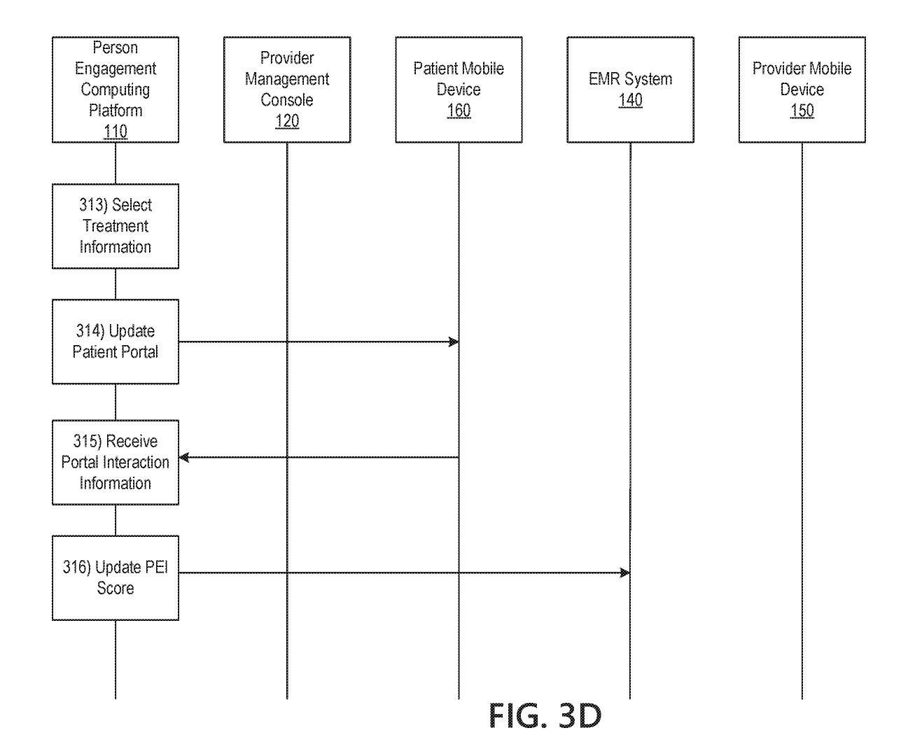

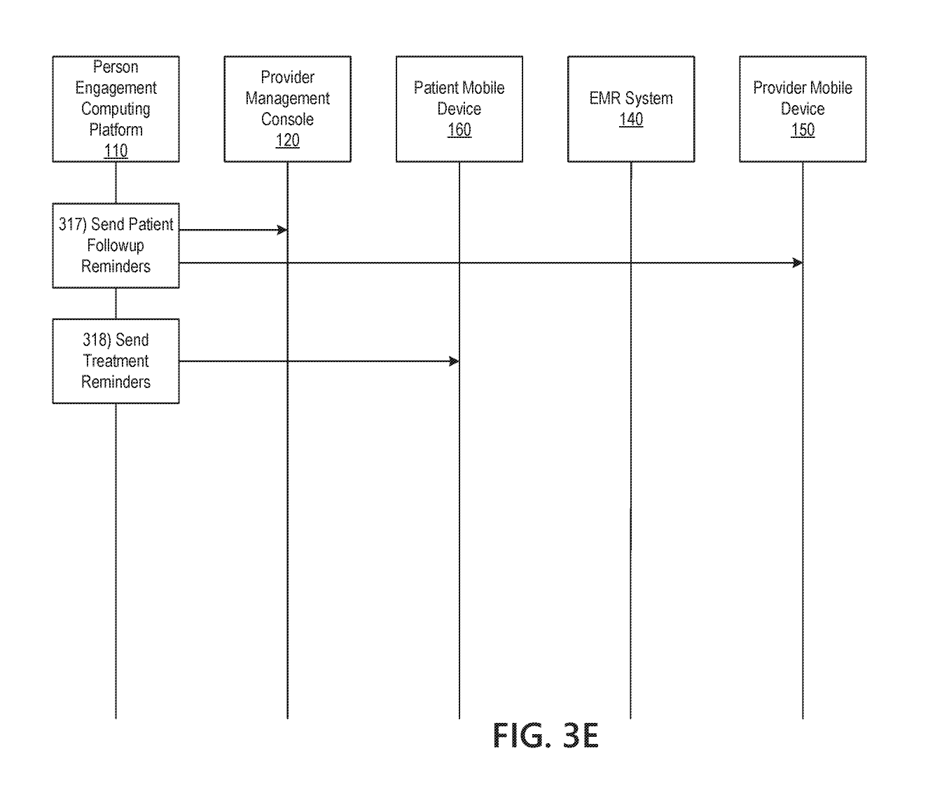

Techniques according to the present disclosure may also be used in an outpatient or ambulatory facility context. In such a context, a patient may not stay at a healthcare facility for a large amount of time (or at all), and accordingly certain procedures such as nurse rounding may be inapplicable. FIGS. 3A-3E illustrate an example process for applying techniques according to the present disclosure in such contexts.

Referring to FIG. 3A, at step 301, person engagement computing platform 110 receives patient information from provider management console 120. For example, during a patient check-in procedure, clinicians and/or administrative staff may determine the patient's name and/or some other patient identifier, which may be entered into provider management console 120. The staff may additionally collect other patient information such as one or more symptoms, a reason for a visit, contact information, and other such patient information, which may be entered into provider management console 120 and/or determined or retrieved from other systems by provider management console 120. The provider management console 120 may communicate some or all of the patient information to person engagement computing platform 110.

At step 302, person engagement computing platform 110 may send a request that causes the EMR system 140 to retrieve a record corresponding to the patient identified by the patient information. The record may contain additional information about the patient from previous visits to the same medical facility or to another medical facility. FIG. 4 illustrates an example patient record 400 containing information such as a patient photo, patient information including a name, date of birth, relatives, and contact information. The record may also include symptoms, diagnoses, procedures, treatments, and other such information about current and past medical issues for the patient. The record and/or patient information contained in the record may be retrieved by EMR system 140 and communicated to person engagement computing platform 110, as illustrated.

In some embodiments, the patient record 400 may include a PEI field 410 for storing a PEI score in the patient record 400. In some instances, at step 302, the patient record 400 may already store a PEI score determined at a previous time (e.g., during a previous visit to a same or different medical facility). In these instances, the EMR system 140 may retrieve the stored PEI score 410 and transmit it to person engagement computing platform 110 at step 202.

At step 303, person engagement computing platform 110 and/or PEI calculation module 113a may estimate an initial person engagement index (PEI) score based on preliminary information. In some embodiments, if patient record 400 already contained a PEI score 410, person engagement computing platform 110 may use the stored PEI score 410 as the initial PEI score. An estimated initial PEI score may, in some embodiments, be determined based on one or more items of patient information obtained in steps 301 and/or 302, such as demographic information, a preferred contact method, a number of relatives, and other such patient information. For example, person engagement computing platform 110 may compare the patient demographic information to demographic information and associated PEI scores for other patients in order to estimate the patient's initial PEI score. Accordingly, demographic information and PEI scores for other patients may, in some embodiments, be stored in memory 112 of person engagement computing platform 110.

In some embodiments, person engagement computing platform 110 and/or PEI calculation module 113a may implement statistical and/or machine learning techniques to train one or more models to estimate an initial PEI score based on demographic information and PEI information for other patients. For example, person engagement computing platform 110 may train a regression model to estimate an initial PEI score based on patient demographic information using training data, and such training data may include corresponding demographic information and PEI scores for other patients. Other such techniques and/or types of models may be used by person engagement computing platform 110 to estimate the initial PEI score.

The PEI score may be determined (e.g., by person engagement computing platform 110) based on several PEI factor sub-scores indicating, for example, a technology use in healthcare factor of the PEI score. The PEI score may be a weighted average of the PEI factor sub-scores. Other patient information, such as preferred contact method, may affect a particular PEI factor sub-score (and correspondingly the PEI score). For example, a preferred contact method of “text message” or “email” may indicate a relatively high technology use in healthcare sub-score as compared to a “telephone” preferred contact method. Similarly, other patient information, such as a number of relatives, may indicate a relatively higher psychosocial support sub-score as compared to fewer relatives. Accordingly, the initial PEI score may be based on various types of patient information.

In some embodiments, person engagement computing platform 110 and/or PEI calculation module 113a may implement statistical and/or machine learning techniques to train one or more models to estimate an initial PEI score based on such additional information (e.g., a number of relatives, a preferred contact method, and the like) and PEI information for other patients. For example, person engagement computing platform 110 may train a regression model to estimate an initial PEI score based on such additional information (together with or separately from demographic information) using training data comprising corresponding additional information (together with or separately from demographic information) and PEI scores for other patients. Other such techniques and/or types of models may be used (e.g., by person engagement computing platform 110) to estimate the initial PEI score.

After determining the initial PEI score, person engagement computing platform 110 may cause the initial PEI score to be displayed at one or more of provider management console 120, patient bedside device 130, provider mobile device 150, and patient mobile device 160.

At step 304, person engagement computing platform 110 may establish a communication session with the patient mobile device 160. In some embodiments, patient information (e.g., as received at step 301 or at another time) may include an indicator of the patient mobile device 160 associated with the patient. Accordingly, person engagement computing platform 110 may send (e.g., via communication interface 114) a request to the indicated patient mobile device 160, which may reply in order to establish the session, as illustrated. In some embodiments, the patient mobile device 160 may send a request to establish a communication session to the person engagement computing platform 110. Such a request received from patient mobile device 160 may include a patient identifier associated with the patient so that person engagement computing platform 110 may associate the session with the patient. The person engagement computing platform 110 may then reply to the request from the patient mobile device 160 in order to establish the session.

Referring to FIG. 3B, at step 305, person engagement computing platform 110 and/or portal generation module 113b may generate a patient portal and deliver the patient portal to the patient bedside device 130. The patient portal may be composed of and/or include one or more user interfaces that may enable and disable options for providing entertainment, educational, and medical information to patients. The person engagement computing platform 110 and/or patient mobile device 160 may customize the patient portal for a particular patient. In some embodiments, the patient portal may be customized in accordance with the initial PEI score. Patient mobile device 160 may display the patient portal and receive inputs from the patient in order to create an interactive experience.

The patient portal may include a link for accessing a survey that may be used to determine a PEI score for the patient. In some situations, the link may be featured prominently within the patient portal. For example, the patient portal may include a dynamic list of links to entertainment and medical content, such as a list of videos chosen for the patient, and a pain management plan tailored to the patient. Such a dynamic list may feature the link to the survey when a patient has not yet completed some or all of a survey for determining a PEI score.

Accordingly, at step 306, the person engagement computing platform 110 and/or portal generation module 113b may be configured to provide a survey to the patient. In some embodiments, the survey is provided via the patient mobile device 160. For example, the patient mobile device 160 may be configured (e.g., by one or more commands received from person engagement computing platform 110) to access, upon a patient selecting a link of the patient portal, a web page or other user interface generated by person engagement computing platform 110 (and/or another device, such as a third party survey tool) for interacting with the survey. Accordingly, in some embodiments, the survey may be rendered within a browser executed by the patient mobile device 160. In some embodiments, the survey may be rendered in some other application executed by the patient mobile device 160. In some instances, if the patient does not select the survey link, the person engagement computing platform 110 may proactively send a notification to the patient mobile device 160, and the notification may prompt the patient to a take the survey. In some embodiments, person engagement computing platform 110 may select the mechanism for delivering the notification based on the initial PEI score. For example, if the initial PEI score indicates a relatively low technology use in healthcare sub-score, the notification may be delivered via a telephone call, whereas if the initial PEI score indicates a relatively high technology use in healthcare sub-score, the notification may be delivered via text message, via email, as a notification delivered via an application of the patient bedside device 130, or via some other mechanism. Accordingly, a PEI score may be used (e.g., by person engagement computing platform 110) to determine a preferred contact method and deliver notifications accordingly. Such a preferred contact method may be stored in a record 400 and/or displayed for the healthcare provider (e.g., at provider management console 120).

In some embodiments, the survey may be delivered via an application on a provider mobile device 150. For example, a provider mobile device 150 may execute an application for mobile patient rounding and data collection. Such an application may integrate with person engagement computing platform 110 to display a survey in addition or in place of the survey delivered via patient portal 500. In some embodiments, a clinician or healthcare provider may administer the survey via provider mobile device 150 based on a patient's ability and/or willingness to take a survey via the patient bedside device 130. Accordingly, person engagement computing platform 110 may deliver the survey via the provider mobile device 150 depending on a condition of the patient. In some instances, person engagement computing platform 110 may deliver the survey via the patient mobile device 160 when the patient is associated with a low initial PEI score.

In some embodiments, person engagement computing platform 110 may provide an alternate mechanism for delivering the survey. For example, the survey may be provided via an automated telephone system. In some embodiments, the person engagement computing platform 110 may deliver the survey via an automated telephone system if a patient is associated with a relatively low PEI score and/or technology use in healthcare sub-score of the PEI score.

FIG. 6 illustrates an example prompt 600 that may be rendered on patient mobile device 160 in response to receiving a notification from person engagement computing platform 110. The person engagement computing platform 110 and/or patient mobile device 160 may cause the display of the prompt 600 at the patient mobile device 160 when a patient has not yet started the survey. The prompt 600 may contain links or functions for beginning the survey and/or postponing the survey. In some embodiments, when a patient still has not taken the survey after the patient mobile device 160 has displayed the prompt 600, person engagement computing platform 110 and/or patient mobile device 160 may cause the patient mobile device 160 to again display prompt 600. In some embodiments, person engagement computing platform 110 and/or patient mobile device 160 may cause the prompt 600 to display repeatedly at a frequency determined (e.g., by person engagement computing platform 110) based on the initial PEI score. For example, a patient with an initial PEI score indicating a relatively low engagement in healthcare sub-score (e.g., less than a threshold score or sub-score) may be prompted less frequently (e.g., in order to avoid bothering the patient) than a patient with a higher engagement in healthcare sub-score.

In some embodiments, person engagement computing platform 110 may send a notification to a provider mobile device 150 associated with a clinician or healthcare provider based on the initial PEI score. For example, when an initial PEI score indicates a relatively low technology use in healthcare sub-score (e.g., less than a threshold score or sub-score), a notification may be sent to the provider mobile device 150 indicating that the patient may need assistance in accessing and completing the survey.

FIG. 7A illustrates an example user interface 700 that patient mobile device 160 may present upon receiving an indication that the patient wishes to begin the survey. As illustrated, the user interface 700 may provide a function for selecting a language for taking the survey. FIG. 7B illustrates an example user interface 710 for presenting one of the questions of the survey. The patient mobile device 160 may present a series of questions in turn and receive corresponding answers. As illustrated, the questions may be presented and/or the answers may be received according to a five-point Likert scale. However, other rating scales may be used in other embodiments. FIG. 7C illustrates a user interface 720 that patient mobile device 160 may present upon receiving answers to all of the questions of the survey. As illustrated, the user interface 720 may include links to access other functions of the patient mobile device 160, such as entertainment functions or other user interfaces.

FIG. 7D illustrates a first example workflow for surveying a patient according to step 206. This workflow and/or the other example workflows discussed herein may be directed by person engagement computing platform 110 and/or performed according to user interfaces generated by and/or otherwise provided by person engagement computing platform 110. As illustrated, patient mobile device 160 may present a patient portal featuring a link for taking the survey. Additionally or alternatively, the patient mobile device 160 may regularly display a prompt to take the survey when the patient has not yet finished the survey. When the patient accesses the survey, the patient mobile device 160 may display the user interface 700, followed by the user interface 710 (e.g., for each question of the survey), followed by the user interface 720.

In some embodiments, the survey may be divided into components or modules, which patient mobile device 160 may present separately (e.g., at different times). Accordingly, patient mobile device 160 may display one or more prompts to take different survey modules. FIGS. 8A, 8B, and 8C illustrate example prompts 800, 810, and 820, respectively. A first example prompt 800 may prompt a patient to take a first survey module pertaining to a technology use in healthcare sub-score of the PEI score. A second example prompt 810 may prompt a patient to take a second survey module pertaining to one or more additional sub-scores of the PEI score. A third example prompt 820 may prompt a patient to take a third survey module pertaining to one or more other sub-scores of the PEI score.

The person engagement computing platform 110 and/or patient mobile device 160 may cause the display of such prompts 800, 810, 820 at patient mobile device 160 when a patient has not yet started the corresponding survey module. The prompts 800, 810, 820 may contain links or functions for beginning the survey module and/or postponing the survey module. In some embodiments, when a patient still has not taken a survey module after patient mobile device 160 has displayed the corresponding prompt 800, 810, 820, person engagement computing platform 110 and/or patient mobile device 160 may cause the patient mobile device 160 to again display the corresponding prompt 800, 810, 820.

In some embodiments, after receiving answers to a first survey module corresponding to a technology use in healthcare sub-score of the PEI score, person engagement computing platform 110 may calculate a partial PEI score and/or a technology use in healthcare sub-score of the PEI score. Based on the partial PEI score and/or PEI factor sub-score, the person engagement computing platform 110 may determine an alternative method for completing the surveying and/or prompting the patient to complete the survey. For example, if the partial PEI score and/or technology use in healthcare sub-score indicates a relatively low use of technology in healthcare (e.g., a score or sub-score falling below a threshold), the person engagement computing platform 110 may switch to an automated telephone system to deliver the remainder of the survey and/or the prompts to complete the survey.

FIG. 8D illustrates an example user interface 830 that patient mobile device 160 may display when a patient completes a survey module displayed on patient mobile device 160. The user interface 830 may include links and/or functions for continuing on to the next survey module and for postponing the next survey module. FIG. 8E illustrates a second example workflow 840 for surveying the patient according to step 306. As illustrated, a patient may access the first survey module by selecting a link displayed in the patient portal. After finishing the first survey module, the patient may continue to the second survey module or postpone the second survey module. If the patient postpones the second survey module, person engagement computing platform 110 and/or patient mobile device 160 may cause patient mobile device 160 to display a prompt 810 that provides access to the second survey module. Additionally or alternatively, a link displayed in the patient portal may provide access to the second survey module after the first survey module is completed. Similarly, after finishing the second survey module, the patient may continue to the third survey module or postpone the third survey module. If the patient postpones the third survey module, person engagement computing platform 110 and/or patient mobile device 160 may cause the patient mobile device 160 to display a prompt 820 that provides access to the third survey module. Additionally or alternatively, a link displayed in the patient portal may provide access to the third survey module after the second survey module is completed. In some embodiments, the survey may be divided into more or fewer than three survey modules. For example, in some embodiments, the survey may be divided into four survey modules, each survey module corresponding to one of the sub-scores of the PEI score.

In some embodiments, each survey module may include one or more questions associated with each PEI factor sub-score of the PEI score. In these embodiments, person engagement computing platform 110 may generate a partial PEI factor sub-score after the patient completes each survey module. The person engagement computing platform 110 may further generate a partial PEI score based on the partial PEI factor sub-scores. Accordingly, patient mobile device 160 may display one or more prompts to take different survey modules. FIGS. 9A, 9B, 9C, and 9D illustrate example prompts 900, 910, 920, and 930, respectively. A first example prompt 900 may prompt a patient to take a first survey module, a second example prompt 910 may prompt the patient to take a second survey module, a third example prompt 920 may prompt the patient to take a third survey module, and a fourth example prompt 930 may prompt the patient to take a fourth survey module.

The person engagement computing platform 110 and/or patient mobile device 160 may cause the display of such prompts 900, 910, 920, 930 by patient mobile device 160 when a patient has not yet started the corresponding survey module. The prompts 900, 910, 920, 930 may contain links or functions for beginning the survey module and/or postponing the survey module. In some embodiments, when a patient still has not taken a survey module after patient mobile device 160 has displayed the corresponding prompt 900, 910, 920, 930, person engagement computing platform 110 and/or patient mobile device 160 may cause patient mobile device 160 to again display the corresponding prompt 900, 910, 920, 930.

FIG. 9E illustrates a third example workflow 940 for surveying the patient according to step 306. As illustrated, a patient may access the first survey module by selecting a link displayed in the patient portal. After finishing the first survey module, the patient may continue to the second survey module or postpone the second survey module. If the patient postpones the second survey module, person engagement computing platform 110 and/or patient mobile device 160 may cause patient mobile device 160 to display a prompt 910 that provides access to the second survey module. Additionally or alternatively, a link displayed in the patient portal may provide access to the second survey module after the first survey module is completed. Similarly, after finishing the second survey module, the patient may continue to the third survey module or postpone the third survey module. If the patient postpones the third survey module, person engagement computing platform 110 and/or patient mobile device 160 may cause patient mobile device 160 to display a prompt 920 that provides access to the third survey module. Additionally or alternatively, a link displayed in the patient portal may provide access to the third survey module after the second survey module is completed. Finally, after finishing the third survey module, the patient may continue to the fourth survey module or postpone the fourth survey module. If the patient postpones the fourth survey module, person engagement computing platform 110 and/or patient mobile device 160 may cause patient mobile device 160 to display a prompt 930 that provides access to the fourth survey module. Additionally or alternatively, a link displayed in the patient portal may provide access to the fourth survey module after the third survey module is completed.

Returning to FIG. 3B, after the patient completes all or part of the survey at step 306, person engagement computing platform 110 and/or PEI calculation module 113a may calculate a PEI score at step 307 based on the survey answers provided. The person engagement computing platform 110 may determine the PEI score based on four PEI factors, including an engagement in healthcare factor, a technology use in healthcare factor, a proactive approach to healthcare factor, and a psychosocial support for healthcare factor. The person engagement computing platform 110 may assign a numeric score to each answer based on the selected answer option. For example, on the example user interface 710, a patient has selected an “Agree” answer, which may correspond to a numeric score of 4. In this example, the “Strongly Agree” answer may correspond to a numeric score of 3, the “Neutral” answer may correspond to a numeric score of 3, the “Disagree” answer may correspond to a numeric score of 2, and the “Strongly Disagree” answer may correspond to a numeric score of 1. However, other such correlations between answers and numeric scores may be used by person engagement computing platform 110 to determine the numeric score corresponding to each answer.

The person engagement computing platform 110 and/or PEI calculation module 113a may additionally determine which PEI factor each answer is associated with. In some embodiments, each question/answer may be associated with a single PEI factor. In other embodiments, some questions/answers may be associated with multiple PEI factors. The person engagement computing platform 110 may subsequently add up the numeric scores corresponding to a PEI factor to yield a raw factor score. Subsequently, the raw factor score may be normalized according to a scale (e.g., a 0-100 scale) to yield a PEI factor sub-score. The person engagement computing platform 110 may calculate the PEI score based on an average of the raw domain scores and/or PEI factor sub-scores. In some embodiments, the average may be a weighted average. The PEI score may be normalized according to a scale (e.g., a 0-100 scale).

In some embodiments, at step 307, person engagement computing platform 110 and/or PEI calculation module 113a may not have received all the answers (e.g., because a patient only completed a portion of the survey or less than all of the survey modules). In these instances, person engagement computing platform 110 may determine a partial PEI score based on the answers that were received. For example, when person engagement computing platform 110 has only received 3 answers associated with the engagement in healthcare PEI factor (e.g., out of a total of 9 on the survey), person engagement computing platform 110 may determine an average numeric score for the 3 answers that were received. Such an average numeric score may be normalized according to a scale (e.g., a 0-100) to determine a partial PEI sub-score. After determining multiple partial PEI sub-scores, person engagement computing platform 110 may determine a partial PEI score based on an average of the partial PEI sub-scores. In some embodiments, the averages may be weighted averages.

In some embodiments, person engagement computing platform 110 and/or PEI calculation module 113a may determine that insufficient answers have been received to generate a partial PEI sub-score. For example, person engagement computing platform 110 may have received no answers, or very few answers (e.g., less than 33% of the answers for a PEI factor) for a particular PEI factor. In these instances, person engagement computing platform 110 may use an estimated PEI factor sub-score (e.g., as determined at step 203) in place of determining a partial PEI factor sub-score for a PEI factor. Accordingly, person engagement computing platform 110 may determine the partial PEI score based in part on survey answers and based in part on demographic information or other information used to estimate a PEI factor sub-score.

After determining the PEI score at step 307, person engagement computing platform 110 may cause the PEI score to be displayed at one or more of provider management console 120, patient bedside device 130, provider mobile device 150, and patient mobile device 160.

At step 308, person engagement computing platform 110 may optionally receive information about a patient treatment plan. The information about the patient treatment plan may be received from a provider management console 120, a provider mobile device 150, or some other computing system associated with a clinician or healthcare provider. The patient treatment plan may include information such as a medication schedule (e.g., time when the patient is scheduled to take particular medications, patient goals (e.g., the clinician may give the patient a goal of walking a certain distance each day to aid recovery), clinicians assigned to the patient (e.g., a clinician that a patient may contact with questions), and other such treatment information.

The person engagement computing platform 110 and/or patient treatment plan module 113c may provide feedback and/or recommendations regarding aspects of the patient treatment plan based on the PEI score. At step 309, in some embodiments, person engagement computing platform 110 may provide a recommendation associated with a patient medication schedule based on the PEI score. In some instances, a low PEI score (or a low engagement in healthcare sub-score and/or a low proactive approach to healthcare sub-score) may tend to indicate that the patient may be less willing to follow and complete a patient medication schedule and/or may be less willing to take multiple medications. For such a patient, person engagement computing platform 110 may recommend modifying patient medication schedule so that the patient takes multiple medications at the same time in order to reduce the number of times the patient must remember to take medication. For patients with higher PEI scores, the person engagement computing platform 110 may avoid recommending that a patient medication schedule should be modified so that the patient takes medications at the same time. Accordingly, person engagement computing platform 110 may recommend one or more modifications to the patient medication schedule based on the PEI score. Such recommendations may be provided to the provider management console 120 and/or other computing system associated with the healthcare provider.

In some embodiments, person engagement computing platform 110 and/or patient treatment plan module 113c may use one or more models (e.g., machine learning models, statistical models, and the like) to analyze the patient medication schedule with respect to the PEI score in order to generate one or more recommendations regarding the patient medication schedule. Such models may have been trained (e.g., by person engagement computing platform 110 or another computing system) based on historical data correlating PEI scores to patient medication schedule compliance. Such a trained model may output an effectiveness score for the patient medication schedule and/or one or more recommendations for improving the effectiveness score. In some embodiments, person engagement computing platform 110 may use one or more rules to generate recommendations. For example, a rule may indicate that for patients with a PEI score below a certain threshold, the patient medication schedule should not require the patient to take medications more than a certain number of times in a given time period (e.g., more than twice daily).

In addition to or as an alternative to providing recommendations about the patient medication schedule, the person engagement computing platform 110 and/or patient treatment plan module 113c may display an effectiveness score pertaining to the patient medication schedule. The person engagement computing platform 110 may generate such an effectiveness score based on the PEI score using trained models and/or rules. The person engagement computing platform 110 may cause the effectiveness score to be displayed on a device associated with a clinician or healthcare provider (e.g., the provider management console), and update the effectiveness score based on changes or modifications to the patient medication schedule received from the clinician or healthcare provider.

At step 310, person engagement computing platform 110 and/or patient treatment plan module 113c may determine a patient reminder schedule based on the PEI score. In some embodiments, a low PEI score (or a low engagement in healthcare sub-score and/or a low proactive approach to healthcare sub-score) may indicate that a patient needs more reminders in order to accomplish tasks such as taking medications, achieving patient goals, and performing other tasks. In contrast, a higher PEI score (or a higher engagement in healthcare sub-score and/or a higher proactive approach to healthcare sub-score) may indicate that the patient does not need frequent reminders in order to accomplish tasks. Accordingly, person engagement computing platform 110 may recommend providing more or fewer notifications for patient tasks such as taking medications, accomplishing goals, and other patient tasks based on the PEI score.

In some embodiments, person engagement computing platform 110 and/or patient treatment plan module 113c may use one or more models (e.g., machine learning models, statistical models, and the like) to analyze a patient reminder schedule with respect to the PEI score in order to generate one or more recommendations regarding the patient reminder schedule. Such models may have been trained (e.g., by person engagement computing platform 110 or another computing system) based on historical data correlating PEI scores to patient task compliance. Such a trained model may output an effectiveness score for the patient reminder schedule and/or one or more recommendations for improving the effectiveness score. In some embodiments, person engagement computing platform 110 may use one or more rules to generate recommendations. For example, a rule may indicate that for patients with a PEI score below a certain threshold, the patient reminder schedule should remind a patient to complete a task a certain number of times in a certain time period (e.g., at least once daily).

In addition to or as an alternative to providing recommendations about the patient reminder plan, the person engagement computing platform 110 and/or patient treatment plan module 113c may display an effectiveness score pertaining to the patient reminder schedule. The person engagement computing platform 110 may generate such an effectiveness score based on the PEI score using trained models and/or rules. The person engagement computing platform 110 may cause the effectiveness score to be displayed on a device associated with a clinician or healthcare provider (e.g., the provider management console), and update the effectiveness score based on changes or modifications to the patient reminder schedule received from the clinician or healthcare provider.

At step 311, person engagement computing platform 110 and/or patient treatment plan module 113c may provide an overall treatment plan effectiveness rating. For example, person engagement computing platform 110 may calculate an average based on the effectiveness ratings for the patient medication schedule and the patient reminder schedule. Accordingly, person engagement computing platform 110 may determine a personalized and comprehensive effectiveness rating of the treatment the patient will receive using the PEI score. The person engagement computing platform 110 may further cause the score to be displayed at a device associated with a clinician or healthcare provider (e.g., the provider management console 120).

At step 312, person engagement computing platform 110 and/or patient treatment plan module 113c may update any aspect of the patient treatment plan based on modification or updates received from a clinician or healthcare provider. If no such modifications or updates are received, person engagement computing platform 110 may confirm the treatment plan received at step 312.

Referring to step FIG. 3D, at step 313, person engagement computing platform 110 may select one or more items of personalized educational content based on the patient information, the patient treatment plan, and the PEI score. The person engagement computing platform 110 may select educational content pertaining to one or more conditions, illnesses, symptoms, medications, treatments, or procedures associated with the patient. Such educational content may include videos, images, audio, and/or text stored in content database 113d.

In some embodiments, person engagement computing platform 110 may select one or more items of educational content from a content database 113d of educational content based on matches between the one or more conditions, illnesses, symptoms, medications, treatments, or procedures associated with the patient and the educational content in the content database 113d. For example, the educational content in the content database 113d may be tagged with keywords, topics, or other metadata that may be compared (e.g., by person engagement computing platform 110) to the one or more conditions, illnesses, symptoms, medications, treatments, or procedures associated with the patient in order to determine a match and select the matched educational content. In some embodiments, person engagement computing platform 110 may implement or use a search engine to select educational content from the content database 113d using queries formulated based on the one or more conditions, illnesses, symptoms, medications, treatments, or procedures associated with the patient. In some embodiments, the matched content may be associated with a rank score (e.g., to yield an ordered list of educational content) based on how closely the content matches the one or more conditions, illnesses, symptoms, medications, treatments, or procedures associated with the patient.

In some embodiments, person engagement computing platform 110 may adjust the rank scores of the selected content based on the PEI score. For example, for patients with a lower PEI score, more engaging content such as videos, images, and audios may be preferred. Accordingly, person engagement computing platform 110 may reduce the rank score of less engaging content such as textual articles for patients with lower PEI scores. Similarly, person engagement computing platform 110 may increase a rank score for more engaging content for patients with lower PEI scores. For patients with higher PEI scores, person engagement computing platform 110 may increase a rank score of more detailed and/or more informative content. The person engagement computing platform 110 may store and execute one or more rules for increasing or decreasing the rank score of content based on the PEI score and based on the content type (e.g., video, images, text, etc.), the level of detail associated with the content (e.g., a content length, Flesch Reading Ease score associated with the content, or some other measure of the level of detail of the content), or other content categories or metrics. For example, person engagement computing platform 110 may store and execute a rule that decreases rank scores of items of educational content by a certain amount for patients with a PEI score above and/or below certain thresholds.

In some embodiments, education content in the content database 113d may be tagged with a target PEI score. For example, a very detailed article about advanced treatment options for a condition may be tagged with a relatively high target PEI score of 100, whereas a more engaging video explaining the basics of a condition may be associated with a relatively moderate target PEI score of 60. Based on the difference between the patient's PEI score and the target PEI score for an item of educational content, the rank score of the item may be adjusted so that educational content with a target PEI score close to the patient's PEI score may be ranked relatively more highly in comparison to educational content with a target PEI score having a large difference with the patient's PEI score.

Accordingly, person engagement computing platform 110 may determine a ranked list of educational content based on patient information, the patient treatment plan, and the PEI score at step 313. At step 314, person engagement computing platform 110 and/or portal generation module 113b may generate one or more commands configured to update the patient portal and/or other aspects of the patient mobile device 160 based on the results of steps 311 and/or 312, and person engagement computing platform 110 may send the one or more commands to the patient mobile device 160. The patient portal may contain a dynamic listing or arrangement of links that are personalized for the patient. In some instances, the person engagement computing platform 110 may generate one or more commands that enable a link for accessing one or more items of personalized educational content. In some examples, the link may access video content selected according to step 313. In some embodiments, a link displayed via the patient portal may access other types of educational content together with or separately from video content. In some embodiments, the person engagement computing platform 110 may generate one or more commands that configure the patient portal to include different links for accessing different types of personalized educational content. Upon receiving a selection of one of the links, the patient mobile device 160 may display a plurality of options to select one or more items of educational content. In some embodiments, the person engagement computing platform 110 may generate one or more commands that configure the patient mobile device 160 to display the plurality of options arranged according to a rank score associated with the respective educational content.

The person engagement computing platform 110 and/or portal generation module 113b, at step 314, may generate one or more commands that update the patient portal to include patient treatment plan information. For example, the one or more commands may enable a link displayed in the patient portal to provide access to a pain management feature of the patient treatment plan, which may include information from the patient medication schedule, as well as other patient treatment plan information, such as educational content about pain management and other content related to pain management. Accordingly, person engagement computing platform 110 may configure the patient mobile device 160 to display information about aspects of the personalized patient treatment plan together with and/or separately from one or more items of personalized educational content.

The person engagement computing platform 110 may generate one or more commands that configure other aspects of the patient portal and/or patient mobile device 160. In some embodiments, person engagement computing platform 110 may generate one or more commands that configure a patient whiteboard (which may be selectable via the patient portal or otherwise may be displayed on patient mobile device 160) to display information from the personalized patient treatment plan. For example, person engagement computing platform 110 may generate one or more commands that configure the patient whiteboard to display a schedule indicating times of scheduled procedures and other items scheduled for the patient according to the patient treatment plan. In some embodiments, the one or more commands may configure the patient whiteboard to display one or more patient goals. In some embodiments, the one or more commands may configure the patient whiteboard to provide forms for allowing the patient to communicate with one or more clinicians assigned to the patient. The person engagement computing platform 110 may generate one or more commands that configure the patient portal to display a link that causes the patient mobile device 160 to display the patient whiteboard. In some embodiments, the person engagement computing platform 110 may generate one or more commands that cause the patient mobile device 160 to enable or disable notifications related to a surveys (and/or modules of the survey), a patient treatment plan, a patient medication schedule, and/or a patient reminder schedule. In some embodiments, the person engagement computing platform 110 may generate one or more commands configured to cause the patient bedside device 130 to display a PEI score. Accordingly, the person engagement computing platform 110 may generate one or more configuration commands for the patient mobile device 160 based on the PEI score associated with the patient. Subsequently, the person engagement computing platform 110 may send the one or more configuration commands to the patient mobile device 160. The patient mobile device 160 may, based on the one or more configuration commands, selectively enable or disable one or more functions of the patient mobile device 160 based on the PEI score associated with the patient.

At step 315, person engagement computing platform 110 may receive content interaction information from the patient mobile device 160. The patient mobile device 160 may monitor a patient's interactions with the personalized educational content accessible via the patient portal and generate the content interaction information based on the monitored interactions. For example, the patient mobile device 160 may track which educational content items the patient selects, how long the patient spends viewing each educational content item, and the like. In some embodiments, patient mobile device 160 may track start and end times associated with video content. For example, patient mobile device 160 may determine which portions of a video content item were viewed, whether a patient skipped certain portions, whether a patient ended a video early, and the like by storing timestamps indicating that a patient began watching a video at a certain point (e.g., by pressing play or by skipping to the point) and stopped the video at another point (e.g., by skipping away at the point, by stopping at the point, etc.). Accordingly, patient mobile device 160 may track one or more start and end timestamps for video educational content. Any and/or all of the information tracked by patient mobile device 160 may be included in the content interaction information. In addition, the patient mobile device 160 may periodically transmit the content interaction information to person engagement computing platform 110.

At step 316, based on the received content interaction information, person engagement computing platform 110 and/or PEI calculation module 113a may optionally update the PEI score. In some embodiments, person engagement computing platform 110 may raise a PEI score based on content interaction information showing a high engagement with the educational content. For example, based on a patient with a relatively low PEI score viewing detailed and/or advanced educational content, the patient's PEI score may be adjusted upwards. In some embodiments, person engagement computing platform 110 may implement rules to adjust the PEI score based on PEI score thresholds, a content type, a level of detail associated with the content, and/or other educational content scores or metrics. An example rule may indicate that a PEI score of less than 60 may be increased by 5 points if the patient spends at least 10 minutes viewing video educational content, and may be increased by 10 points if the patient spends at least 20 minutes viewing textual educational content with a high level of detail. As another example, a rule may provide a formula for adjusting a PEI score upwards in proportion to time spent viewing educational content and the average level of detail associated with the viewed educational content. In some embodiments, person engagement computing platform 110 may use one or more rules for lowering a PEI score based on content interaction information showing low engagement with educational content. For example, if a patient fails to complete viewing one or more items of video educational content, a PEI score may be lowered. The person engagement computing platform 110 may execute such rules and update the PEI score accordingly.

In some embodiments, person engagement computing platform 110 and/or PEI calculation module 113a may adjust the PEI score based on a target PEI score associated with educational content indicated in the content interaction information. For example, if a patient with a PEI score of 70 views an item of educational content with a target PEI score of 80, the patient's PEI score may be adjusted upwards. In some embodiments, person engagement computing platform 110 may calculate the average target PEI score of several items of educational content, and adjust a PEI score (e.g., upwards) in proportion to the difference between the average target PEI score and patient's PEI score, such that viewing educational content with a higher target PEI score may cause the patient's PEI score to be raised by a relatively higher amount. After adjusting and/or updating the PEI score at step 316, person engagement computing platform 110 may cause the updated PEI score to be displayed at one or more of provider management console 120, patient bedside device 130, provider mobile device 150, and patient mobile device 160.

Referring to FIG. 3E, at step 317, person engagement computing platform 110 may send one or more reminders to devices associated with clinicians or healthcare providers, such as provider management console 120 and provider mobile device 150, in accordance with the patient treatment plan. In some instances, the one or more reminders may remind the clinician, and/or healthcare provider about scheduled items, such as a scheduled dose of medication for the patient. In some instances, the one or more reminders may remind the clinician, and/or healthcare provider about one or more patient goals. In some embodiments, reminders may be sent to one or more relatives of the patient (e.g., as determined from the EMR system 140 at step 302). In some embodiments, person engagement computing platform 110 may send reminders to relatives of the patient when the patient is associated with a low PEI score (and/or a low engagement in healthcare and/or proactive approach to healthcare sub-score).

At step 318, person engagement computing platform 110 may send one or more reminders to the patient mobile device 160 (e.g., reminders to take medications, reminders to schedule and/or attend follow-up procedures, and the like). In some embodiments, the reminders may be sent to one or more relatives of the patient (e.g., as determined from the EMR system 140 in step 302) in addition to or as an alternative to the patient. In some embodiments, person engagement computing platform 110 may send reminders to relatives of the patient when the patient is associated with a low PEI score (and/or a low engagement in healthcare and/or proactive approach to healthcare sub-score).

In addition to or as an alternative to one or more of the steps performed according to the illustrative event sequences of FIGS. 2A-2F, FIGS. 3A-3E, and/or any other event sequence, method, or process implementing the techniques described herein, one or more of the patient engagement computing platform 110, the provider management console 120, the patient bedside device 130, the provider mobile device 150, and/or the patient mobile device 160 may be configured to perform additional functions based on the PEI score. For example, a PEI score may be classified as falling within a range of “high,” “medium,” or “low” based on comparing the PEI score to one or more thresholds, and additional functions may be performed as described in TABLE 1, below.

TABLE 1

| | Exchange | | | |
|---|---|---|---|---|
| | information | Planning | | |
| | and | between | Appropriate | |
| | communicate | person and | interventions | Evaluate |
| PEI Score | choices | clinician | determined | regularly |
| LOW | Assess | Start with | Educational | Incremental |
| | current | small, | topics | improvements |
| | knowledge | manageable, | tailored to | can be |
| | and | goals which | the | evaluated |
| | understanding | can be | individual's | through |
| | of health | achieved. | current | review of |
| | status. | | knowledge | documentation |
| | | | level, | in log. |
| | | | preferences | |
| | | | and values. | |
| MEDIUM | Can describe | Discuss | Accesses | Monitoring |
| | healthcare | options for | resources | one's |
| | status and | healthcare | to assist | progress to |
| | shares | goals. | with | healthcare |
| | credible | | achieving | goals. |
| | information. | | healthcare | |
| | | | goals. | |
| HIGH | Understands | Sets | Shared | Maintaining |
| | healthcare | healthcare | decision | optimal health |
| | options and | goals as | making for | status based |
| | good | part of the | interventions. | on goals. |
| | rationale for | healthcare | | |
| | choices to | team. | | |
| | self-manage | | | |
| | health. | | | |

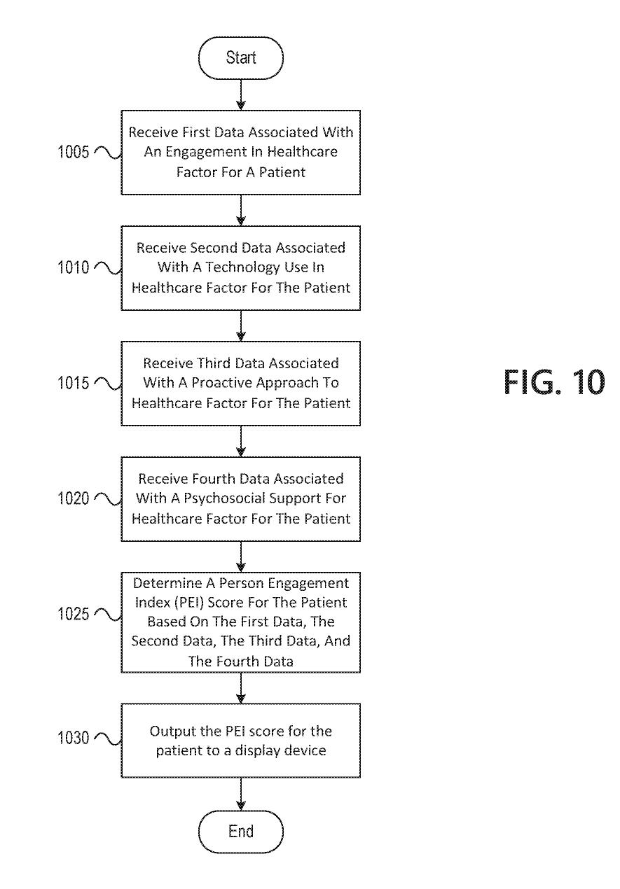

FIG. 10 depicts an illustrative method for performing one or more automated personalized healthcare functions. Referring to FIG. 10, at step 1005, a computing platform having at least one processor, a memory, and a communication interface may receive first data associated with an engagement in healthcare factor for a patient. At step 1010, the computing platform may receive second data associated with a technology use in healthcare factor for the patient. At step 1015, the computing platform may receive third data associated with a proactive approach to healthcare factor for the patient. At step 1020, the computing platform may receive fourth data associated with a psychosocial support for healthcare factor for the patient. At step 1025, the computing platform may determine a person engagement index (PEI) score for the patient based on the first data, the second data, the third data, and the fourth data. At step 1030, the computing platform may output the PEI score for the patient to a display device.

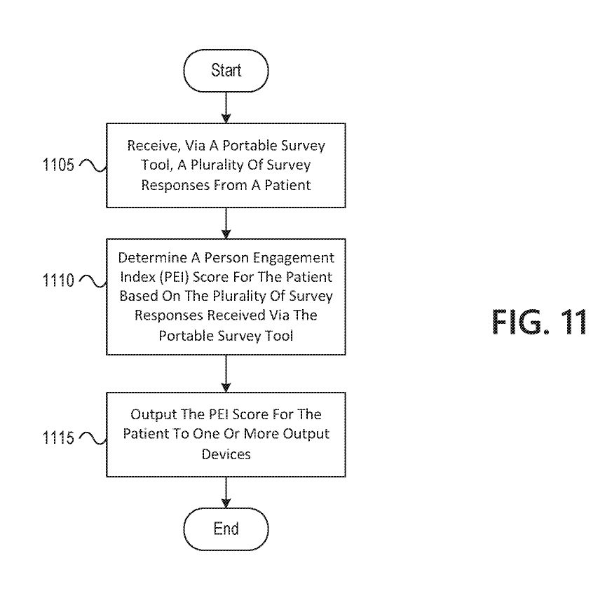

FIG. 11 depicts an illustrative method for performing one or more automated personalized healthcare functions. Referring to FIG. 11, at step 1105, a computing platform having at least one processor, a memory, and a communication interface may receive, via a portable survey tool, a plurality of survey responses from a patient. The plurality of survey responses may comprise first responses indicative of an engagement in healthcare factor for the patient, second responses indicative of a technology use in healthcare factor for the patient, third responses indicative of a proactive approach to healthcare factor for the patient, and fourth responses indicative of a psychosocial support for healthcare factor for the patient. At step 1110, the computing platform may determine a person engagement index (PEI) score for the patient based on the plurality of survey responses received via the portable survey tool. At step 1115, the computing platform may output the PEI score for the patient to one or more output devices.

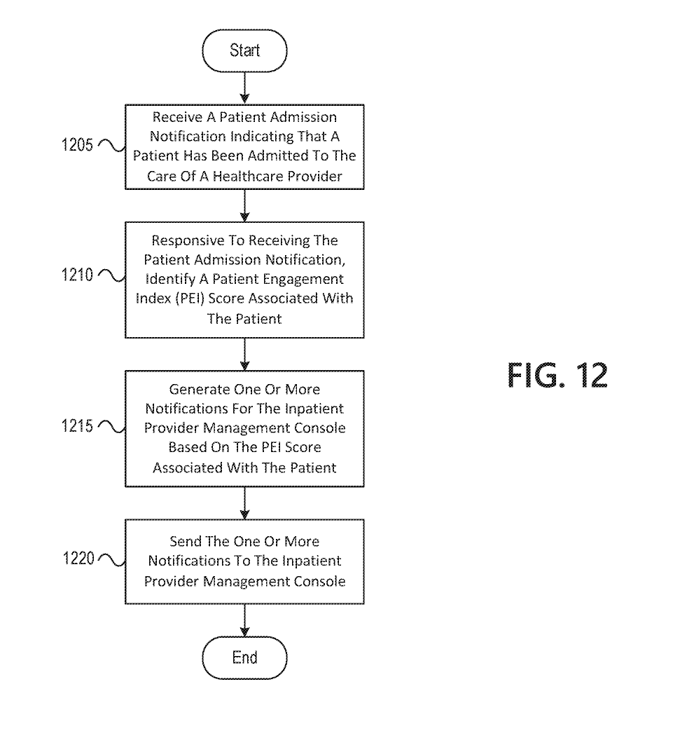

FIG. 12 depicts an illustrative method for performing one or more automated personalized healthcare functions. Referring to FIG. 12, at step 1205, a computing platform having at least one processor, a memory, and a communication interface may receive, from an inpatient provider management console, a patient admission notification indicating that a patient has been admitted to the care of a healthcare provider. At step 1210, responsive to receiving the patient admission notification, the computing platform may identify a person engagement index (PEI) score associated with the patient. At step 1215, the computing platform may generate one or more notifications for the inpatient provider management console based on the PEI score associated with the patient. At step 1220, the computing platform may send the one or more notifications to the inpatient provider management console, wherein the one or more notifications instruct the healthcare provider to perform specific actions based on the PEI score associated with the patient.

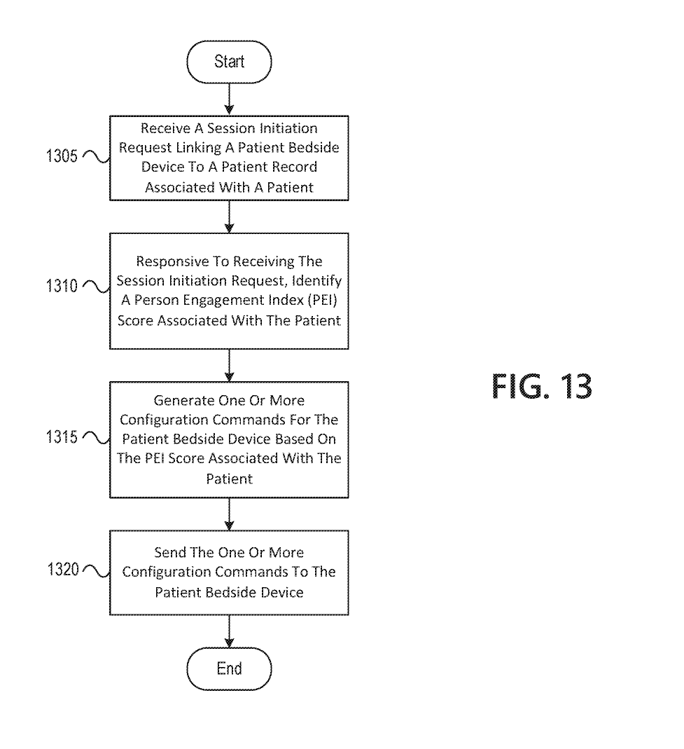

FIG. 13 depicts an illustrative method for performing one or more automated personalized healthcare functions. Referring to FIG. 13, at step 1305, a computing platform having at least one processor, a memory, and a communication interface may receive, from a patient bedside device, a session initiation request linking the patient bedside device to a patient record associated with a patient. At step 1310, responsive to receiving the session initiation request, the computing platform may identify a person engagement index (PEI) score associated with the patient. At step 1315, the computing platform may generate one or more configuration commands for the patient bedside device based on the PEI score associated with the patient. At step 1320, the computing platform may send the one or more configuration commands to the patient bedside device. The one or more configuration commands may direct the patient bedside device to selectively enable or disable one or more functions of the patient bedside device based on the PEI score associated with the patient.

In some arrangements, person engagement computing platform 110 may be configured to extend and/or otherwise provide functionality similar to the functionality described above to adolescent users (e.g., instead of or in addition to adult users) and associated user devices in one or more customized ways. For example, teenage patients and/or other adolescent users may interact with person engagement computing platform 110 via one or more adolescent user computing devices (e.g., smart phones, tablet computers, etc.) in an outpatient clinic setting (e.g., instead of or in addition to in an inpatient hospital setting). The example event sequence flows, the example methods, and/or the example user interface layouts described above may be customized and/or otherwise adapted both to appeal to adolescent users and to operate efficiently on user devices in an outpatient clinic setting accordingly. For instance, the example event sequence flows, methods, and/or user interface layouts may be modified to operate more efficiently and/or effectively when notifications are sent and/or other output is provided to a user device that is not located in a hospital or other inpatient hospital setting (e.g., and is instead located in an outpatient clinic setting) and/or may be optimized to receive user input from a user device that is not located in a hospital or other inpatient hospital setting.

In addition, person engagement computing platform 110 may calculate and/or otherwise determine an adolescent capacity to engage (ACE) index for a particular adolescent user, similar to how person engagement computing platform 110 may determine a PEI score in the examples discussed above. Like a PEI score, an ACE index may reflect a particular person's level of engagement in healthcare and may be determined across the same and/or similar domains, using similar computing technology and/or methods as described above, as a PEI score. For instance, an ACE index may reflect a particular adolescent's participation in healthcare, technology use in healthcare, proactive approach to healthcare, psychosocial support, and/or transition readiness. In calculating and/or otherwise determining an ACE index for a particular adolescent, person engagement computing platform 110 may utilize one or more of the survey questions described above (e.g., for adult patients) and/or one or more different survey questions (which may, e.g., be varied and/or customized for adolescent users based on the particular age of particular adolescent user(s)). In some instances, the fonts, colors, themes, and/or other user interface elements generated and/or presented by person engagement computing platform 110 in presenting survey questions, gathering other user input, and/or presenting output may be varied and/or customized for adolescent users. For instance, certain aspects of one or more user interfaces may be varied and/or customized to better appeal to adolescent users (e.g., instead of adult users). Additionally or alternatively, suggested interventions that may be determined and/or output by person engagement computing platform 110 (e.g., based on an ACE index and/or PEI score) may be the same or may vary for adolescent users. Additionally or alternatively, survey results and/or other processing output determined and/or otherwise generated by person engagement computing platform 110 may be sent to one or more parent user devices (e.g., linked to a particular adolescent user's parents) instead of or in addition to the particular adolescent's user device (e.g., similar to how person engagement computing platform 110 may send processing output and/or other information to a care partner of an adult user in one or more adult-oriented examples discussed above).

One or more aspects of the disclosure may be embodied in computer-usable data or computer-executable instructions, such as in one or more program modules, executed by one or more computers or other devices to perform the operations described herein. Generally, program modules include routines, programs, objects, components, data structures, and the like that perform particular tasks or implement particular abstract data types when executed by one or more processors in a computer or other data processing device. The computer-executable instructions may be stored as computer-readable instructions on a computer-readable medium such as a hard disk, optical disk, removable storage media, solid-state memory, RAM, and the like. The functionality of the program modules may be combined or distributed as desired in various embodiments. In addition, the functionality may be embodied in whole or in part in firmware or hardware equivalents, such as integrated circuits, application-specific integrated circuits (ASICs), field programmable gate arrays (FPGA), and the like. Particular data structures may be used to more effectively implement one or more aspects of the disclosure, and such data structures are contemplated to be within the scope of computer executable instructions and computer-usable data described herein.

Various aspects described herein may be embodied as a method, an apparatus, or as one or more computer-readable media storing computer-executable instructions. Accordingly, those aspects may take the form of an entirely hardware embodiment, an entirely software embodiment, an entirely firmware embodiment, or an embodiment combining software, hardware, and firmware aspects in any combination. In addition, various signals representing data or events as described herein may be transferred between a source and a destination in the form of light or electromagnetic waves traveling through signal-conducting media such as metal wires, optical fibers, or wireless transmission media (e.g., air or space). In general, the one or more computer-readable media may be and/or include one or more non-transitory computer-readable media.

As described herein, the various methods and acts may be operative across one or more computing servers and one or more networks. The functionality may be distributed in any manner, or may be located in a single computing device (e.g., a server, a client computer, and the like). For example, in alternative embodiments, one or more of the computer systems discussed above may be combined into a single computing platform, and the various functions of each computer system may be performed by the single computing platform. In such arrangements, any and/or all of the above-discussed communications between computer systems may correspond to data being accessed, moved, modified, updated, and/or otherwise used by the single computing platform. Additionally or alternatively, one or more of the computer systems discussed above may be implemented in one or more virtual machines that are provided by one or more physical computing devices. In such arrangements, the various functions of each computer system may be performed by the one or more virtual machines, and any and/or all of the above-discussed communications between computer systems may correspond to data being accessed, moved, modified, updated, and/or otherwise used by the one or more virtual machines.

Aspects of the disclosure have been described in terms of illustrative embodiments thereof. Numerous other embodiments, modifications, and variations within the scope and spirit of the appended claims will occur to persons of ordinary skill in the art from a review of this disclosure. For example, one or more of the steps depicted in the illustrative figures may be performed in other than the recited order, and one or more depicted steps may be optional in accordance with aspects of the disclosure.

1. A computing platform comprising:
at least one processor;
a communication interface communicatively coupled to the at least one processor; and
memory storing computer-readable instructions that, when executed by the at least one processor, cause the computing platform to:
receive first data associated with an engagement in healthcare factor for a patient;
receive second data associated with a technology use in healthcare factor for the patient;
receive third data associated with a proactive approach to healthcare factor for the patient;
receive fourth data associated with a psychosocial support for healthcare factor for the patient;
determine a person engagement index (PEI) score for the patient based on the first data, the second data, the third data, and the fourth data; and
output the PEI score for the patient to a display device.

2. The computing platform of claim 1, wherein the first data comprises fifth data associated with a safety involvement sub-factor for the patient, sixth data associated with a health literacy sub-factor for the patient, seventh data associated with a preventative actions sub-factor for the patient, and eighth data associated with a patient preferences sub-factor for the patient.

3. The computing platform of claim 1, wherein the third data comprises ninth data associated with an activation/motivation sub-factor for the patient, tenth data associated with a patient preferences sub-factor for the patient, and eleventh data associated with a psychosocial support sub-factor for the patient.

4. The computing platform of claim 1, wherein at least one of the first data, the second data, the third data, and the fourth data comprises answers to a plurality of survey questions.

5. The computing platform of claim 1, wherein at least one of the first data, the second data, the third data, and the fourth data comprises patient demographic information.

6. The computing platform of claim 1, wherein the memory stores additional computer-readable instructions that, when executed by the at least one processor, cause the computing platform to:
select, from a database, based on the PEI score, educational content for provisioning to the patient; and
transmit the selected educational content to a mobile device associated with the patient.

7. The computing platform of claim 6, wherein at least one of the first data, the second data, the third data, and the fourth data comprises interaction information indicating one or more patient interactions with the selected educational content.

8. The computing platform of claim 1, wherein the memory stores additional computer-readable instructions that, when executed by the at least one processor, cause the computing platform to:
determine a patient treatment plan based on the PEI score associated with the patient; and
output the patient treatment plan to the display device.

9. The computing platform of claim 8, wherein the patient treatment plan comprises a patient medication plan.

10. The computing platform of claim 1, wherein the memory stores additional computer-readable instructions that, when executed by the at least one processor, cause the computing platform to:
determine a preferred contact method for contacting the patient based on the PEI score associated with the patient; and
output the preferred contact method to the display device.

11. The computing platform of claim 1, wherein the memory stores additional computer-readable instructions that, when executed by the at least one processor, cause the computing platform to:
determine, based on the PEI score associated with the patient, an effectiveness of a patient treatment plan; and
output the effectiveness of the patient treatment plan to the display device.

12. A method comprising:
at a computing platform comprising at least one processor, memory, and a communication interface:
receiving, by the at least one processor, first data associated with an engagement in healthcare factor for a patient;
receiving, by the at least one processor, second data associated with a technology use in healthcare factor for the patient;
receiving, by the at least one processor, third data associated with a proactive approach to healthcare factor for the patient;
receiving, by the at least one processor, fourth data associated with a psychosocial support for healthcare factor for the patient;
determining, by the at least one processor, a person engagement index (PEI) score for the patient based on the first data, the second data, the third data, and the fourth data; and
outputting, by the at least one processor, the PEI score for the patient to a display device.

13. The method of claim 12, wherein the first data comprises fifth data associated with a safety involvement sub-factor for the patient, sixth data associated with a health literacy sub-factor for the patient, seventh data associated with a preventative actions sub-factor for the patient, and eighth data associated with a patient preferences sub-factor for the patient.

14. The method of claim 12, wherein the third data comprises ninth data associated with an activation/motivation sub-factor for the patient, tenth data associated with a patient preferences sub-factor for the patient, and eleventh data associated with a psychosocial support sub-factor for the patient.

15. The method of claim 12, wherein the first data, the second data, the third data, and the fourth data comprises one or more of answers to a plurality of survey questions and patient demographic information.

16. The method of claim 12, further comprising:
selecting, by the at least one processor, from a database, based on the PEI score, educational content for provisioning to the patient; and
transmitting, by the at least one processor, via the communication interface, the selected educational content to a mobile device associated with the patient.

17. The method of claim 12, further comprising:
determining, by the at least one processor, a patient treatment plan based on the PEI score associated with the patient; and
outputting, by the at least one processor, the patient treatment plan to the display device.

18. The method of claim 12, further comprising:
determining, by the at least one processor, a preferred contact method for contacting the patient based on the PEI score associated with the patient; and
outputting, by the at least one processor, the preferred contact method to the display device.

19. The method of claim 12, further comprising:
determining, by the at least one processor, based on the PEI score associated with the patient, an effectiveness of a patient treatment plan; and
outputting, by the at least one processor, the effectiveness of the patient treatment plan to the display device.

20. One or more non-transitory computer-readable media comprising instructions that, when executed by a computing platform comprising at least one processor, memory, and a communication interface, cause the computing platform to:
receive first data associated with an engagement in healthcare factor for a patient;
receive second data associated with a technology use in healthcare factor for the patient;
receive third data associated with a proactive approach to healthcare factor for the patient;
receive fourth data associated with a psychosocial support for healthcare factor for the patient;
determine a person engagement index (PEI) score for the patient based on the first data, the second data, the third data, and the fourth data; and
output the PEI score for the patient to a display device.

21. A computing platform comprising:
at least one processor;
a communication interface communicatively coupled to the at least one processor; and
memory storing computer-readable instructions that, when executed by the at least one processor, cause the computing platform to:
receive, via a portable survey tool, a plurality of survey responses from a patient, the plurality of survey responses comprising:
first responses indicative of an engagement in healthcare factor for the patient;
second responses indicative of a technology use in healthcare factor for the patient;
third responses indicative of a proactive approach to healthcare factor for the patient; and
fourth responses indicative of a psychosocial support for healthcare factor for the patient;
determine a person engagement index (PEI) score for the patient based on the plurality of survey responses received via the portable survey tool; and
output the PEI score for the patient to one or more output devices.

22. The computing platform of claim 21, wherein the portable survey tool comprises a mobile application running on a mobile device associated with the patient.

23. The computing platform of claim 21, wherein the portable survey tool comprises a web-based interface provided by a website.

24. The computing platform of claim 21, wherein the memory stores additional computer-readable instructions that, when executed by the at least one processor, cause the computing platform to:
transmit, to a mobile device associated with the patient, a link for accessing the portable survey tool.

25. The computing platform of claim 21, wherein, to receive the plurality of survey responses from the patient, the memory stores additional computer-readable instructions that, when executed by the at least one processor, cause the computing platform to:
receive, via the portable survey tool, at least the first responses;
determine that one or more of the second responses, the third responses, and the fourth responses have not been received; and
transmit, to a mobile device associated with the patient, a link to a website configured to prompt the patient to provide the one or more responses that have not been received.

26. The computing platform of claim 21, wherein the memory stores additional computer-readable instructions that, when executed by the at least one processor, cause the computing platform to:
select, from a database, based on the PEI score, educational content for provisioning to the patient; and
transmit the selected educational content to a mobile device associated with the patient.

27. The computing platform of claim 21, wherein the memory stores additional computer-readable instructions that, when executed by the at least one processor, cause the computing platform to:
determine a patient treatment plan based on the PEI score associated with the patient; and
output the patient treatment plan to the one or more output devices.

28. The computing platform of claim 27, wherein the patient treatment plan comprises a patient medication plan.

29. The computing platform of claim 21, wherein the memory stores additional computer-readable instructions that, when executed by the at least one processor, cause the computing platform to:
determine a preferred contact method for contacting the patient based on the PEI score associated with the patient; and
output the preferred contact method to the one or more output devices.

30. The computing platform of claim 21, wherein the memory stores additional computer-readable instructions that, when executed by the at least one processor, cause the computing platform to:
determine, based on the PEI score associated with the patient, an effectiveness of a patient treatment plan; and
output the effectiveness of the patient treatment plan to the one or more output devices.

31. A method comprising:
at a computing platform comprising at least one processor, memory, and a communication interface:
receiving, by the at least one processor, via a portable survey tool, a plurality of survey responses from a patient, the plurality of survey responses comprising:
first responses indicative of an engagement in healthcare factor for the patient;
second responses indicative of a technology use in healthcare factor for the patient;
third responses indicative of a proactive approach to healthcare factor for the patient; and
fourth responses indicative of a psychosocial support for healthcare factor for the patient;
determining, by the at least one processor, a person engagement index (PEI) score for the patient based on the plurality of survey responses received via the portable survey tool; and
outputting, by the at least one processor, the PEI score for the patient to one or more output devices.

32. The method of claim 31, wherein the portable survey tool comprises one of a mobile application running on a mobile device associated with the patient or a web-based interface provided by a website.

33. The method of claim 31, further comprising:
transmitting, by the at least one processor, to a mobile device associated with the patient, a link for accessing the portable survey tool.

34. The method of claim 31, wherein receiving the plurality of survey responses from the patient comprises:
receiving, via the portable survey tool, at least the first responses;
determining that one or more of the second responses, the third responses, and the fourth responses have not been received; and
transmitting, via the communication interface, to a mobile device associated with the patient, a link to a website configured to prompt the patient to provide the one or more responses that have not been received.

35. The method of claim 31, further comprising:
selecting, by the at least one processor, from a database, based on the PEI score, educational content for provisioning to the patient; and
transmitting, by the at least one processor, via the communication interface, the selected educational content to a mobile device associated with the patient.

36. The method of claim 31, further comprising:
determining, by the at least one processor, a patient treatment plan based on the PEI score associated with the patient; and
outputting, by the at least one processor, the patient treatment plan to the one or more output devices.

37. The method of claim 36, wherein the patient treatment plan comprises a patient medication plan.

38. The method of claim 31, further comprising:
determining, by the at least one processor, a preferred contact method for contacting the patient based on the PEI score associated with the patient; and
outputting, by the at least one processor, the preferred contact method to the one or more output devices.

39. The method of claim 31, further comprising:
determining, by the at least one processor, based on the PEI score associated with the patient, an effectiveness of a patient treatment plan; and
outputting, by the at least one processor, the effectiveness of the patient treatment plan to the one or more output devices.

40. One or more non-transitory computer-readable media comprising instructions that, when executed by a computing platform comprising at least one processor, memory, and a communication interface, cause the computing platform to:
receive, via a portable survey tool, a plurality of survey responses from a patient, the plurality of survey responses comprising:
first responses indicative of an engagement in healthcare factor for the patient;
second responses indicative of a technology use in healthcare factor for the patient;
third responses indicative of a proactive approach to healthcare factor for the patient; and
fourth responses indicative of a psychosocial support for healthcare factor for the patient;
determine a person engagement index (PEI) score for the patient based on the plurality of survey responses received via the portable survey tool; and
output the PEI score for the patient to one or more output devices.

41. A computing platform comprising:
at least one processor;
a communication interface communicatively coupled to the at least one processor; and
memory storing computer-readable instructions that, when executed by the at least one processor, cause the computing platform to:
receive, from an inpatient provider management console, a patient admission notification indicating that a patient has been admitted to the care of a healthcare provider;
responsive to receiving the patient admission notification, identify a person engagement index (PEI) score associated with the patient;
generate one or more notifications for the inpatient provider management console based on the PEI score associated with the patient; and
send the one or more notifications to the inpatient provider management console, wherein the one or more notifications instruct the healthcare provider to perform specific actions based on the PEI score associated with the patient.

42. The computing platform of claim 41, wherein, to identify the PEI score associated with the patient, the memory stores additional computer-readable instructions that, when executed by the at least one processor, cause the computing platform to:
receive patient information associated with the patient; and
determine the PEI score based on the patient information.

43. The computing platform of claim 42, wherein the patient information comprises answers to one or more questions provided to the patient via a survey tool.

44. The computing platform of claim 42, wherein the patient information comprises patient demographic information.

45. The computing platform of claim 42, wherein the patient information comprises interaction information indicating one or more patient interactions with educational content.

46. The computing platform of claim 41, wherein the memory stores additional computer-readable instructions that, when executed by the at least one processor, cause the computing platform to:
determine a patient medication plan based on the PEI score associated with the patient,
wherein the one or more notifications comprise a notification instructing the healthcare provider to provide medication to the patient in accordance with the patient medication plan.

47. The computing platform of claim 41, wherein the memory stores additional computer-readable instructions that, when executed by the at least one processor, cause the computing platform to:
determine a preferred contact method for contacting the patient based on the PEI score associated with the patient,
wherein the one or more notifications comprise a notification indicating the preferred contact method.

48. The computing platform of claim 41, wherein the memory stores additional computer-readable instructions that, when executed by the at least one processor, cause the computing platform to:
determine, based on the PEI score associated with the patient, a reminder frequency for reminding the patient to follow a patient treatment plan,
wherein the one or more notifications comprise a notification indicating that the patient should be reminded to follow the patient treatment plan.

49. The computing platform of claim 41, wherein the memory stores additional computer-readable instructions that, when executed by the at least one processor, cause the computing platform to:
determine, based on the PEI score associated with the patient, an effectiveness of a patient treatment plan,
wherein the one or more notifications comprise a notification indicating the effectiveness of the patient treatment plan.

50. The computing platform of claim 41, wherein, to identify the PEI score associated with the patient, the memory stores additional computer-readable instructions that, when executed by the at least one processor, cause the computing platform to:
transmit, via the communication interface, a patient identifier to an electronic medical records (EMR) system; and
receive, from the EMR system, the PEI score associated with the patient.

51. A method comprising:
at a computing platform comprising at least one processor, memory, and a communication interface:
receiving, by the at least one processor, from an inpatient provider management console, a patient admission notification indicating that a patient has been admitted to the care of a healthcare provider;
responsive to receiving the patient admission notification, identifying, by the at least one processor, a person engagement index (PEI) score associated with the patient;
generating, by the at least one processor, one or more notifications for the inpatient provider management console based on the PEI score associated with the patient; and
sending, by the at least one processor, the one or more notifications to the inpatient provider management console, wherein the one or more notifications instruct the healthcare provider to perform specific actions based on the PEI score associated with the patient.

52. The method of claim 51, wherein identifying the PEI score associated with the patient further comprises:
receiving patient information associated with the patient; and
determining the PEI score based on the patient information.

53. The method of claim 52, wherein the patient information comprises answers to one or more questions provided to the patient via a survey tool.

54. The method of claim 52, wherein the patient information comprises patient demographic information.

55. The method of claim 52, wherein the patient information comprises interaction information indicating one or more patient interactions with educational content.

56. The method of claim 51, further comprising:
determining, by the at least one processor, a patient medication plan based on the PEI score associated with the patient,
wherein the one or more notifications comprise a notification instructing the healthcare provider to provide medication to the patient in accordance with the patient medication plan.

57. The method of claim 51, further comprising:
determining, by the at least one processor, a preferred contact method for contacting the patient based on the PEI score associated with the patient,
wherein the one or more notifications comprise a notification indicating the preferred contact method.

58. The method of claim 51, further comprising:
determining, by the at least one processor, based on the PEI score associated with the patient, a reminder frequency for reminding the patient to follow a patient treatment plan,
wherein the one or more notifications comprise a notification indicating that the patient should be reminded to follow the patient treatment plan.

59. The method of claim 51, further comprising:
determining, by the at least one processor, based on the PEI score associated with the patient, an effectiveness of a patient treatment plan,
wherein the one or more notifications comprise a notification indicating the effectiveness of the patient treatment plan.

60. One or more non-transitory computer-readable media comprising instructions that, when executed by a computing platform comprising at least one processor, memory, and a communication interface, cause the computing platform to:
receive, from an inpatient provider management console, a patient admission notification indicating that a patient has been admitted to the care of a healthcare provider;
responsive to receiving the patient admission notification, identify a person engagement index (PEI) score associated with the patient;
generate one or more notifications for the inpatient provider management console based on the PEI score associated with the patient; and
send the one or more notifications to the inpatient provider management console, wherein the one or more notifications instruct the healthcare provider to perform specific actions based on the PEI score associated with the patient.
